SEQUENCE LISTING (22 sequences)

SEQ ID NO: 1 - DNA, 23 bp, Artificial Sequence
  Description of Artificial Sequence Synthetic primer
ggtacctggaagggctaatttgg

SEQ ID NO: 2 - DNA, 23 bp, Artificial Sequence
  Description of Artificial Sequence Synthetic primer
ggtacctcgagctttctacaagg

SEQ ID NO: 3 - DNA, 23 bp, Artificial Sequence
  Description of Artificial Sequence Synthetic primer
tctagaggaggtgtggcctgggc

SEQ ID NO: 4 - DNA, 23 bp, Artificial Sequence
  Description of Artificial Sequence Synthetic primer
ggtaccagatgctacatataagc

SEQ ID NO: 5 - DNA, 21 bp, Artificial Sequence
  Description of Artificial Sequence Synthetic primer
ccatggtaagcagtgggttcc

SEQ ID NO: 6 - DNA, 560 bp, Artificial Sequence
  Description of Artificial Sequence Synthetic polynucleotide
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtg

SEQ ID NO: 7 - DNA, 395 bp, Artificial Sequence
  Description of Artificial Sequence Synthetic polynucleotide
gatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagc

SEQ ID NO: 8 - DNA, 83 bp, Artificial Sequence
  Description of Artificial Sequence Synthetic oligonucleotide
gatctgtggatctaccacacacaaggctacttccctgattggcagaactacacaccagggccagggatcagatatccactgac

SEQ ID NO: 9 - DNA, 190 bp, Artificial Sequence
  Description of Artificial Sequence Synthetic polynucleotide
ctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggag

SEQ ID NO: 10 - DNA, 205 bp, Artificial Sequence
  Description of Artificial Sequence Synthetic polynucleotide
gatctgtggatctaccacacacaaggctacttccctgattggcagaactacacaccagggccagggatcagatatccactgactactacaaagactgctgacatcgagctttctacaagggactttccgctggggactttccagggaggtgtggcctgggcgggactggggagtggcgagccctcagatgctacatataagcagc

SEQ ID NO: 11 - DNA, 560 bp, Artificial Sequence; identical to SEQ ID NO: 6
  Description of Artificial Sequence Synthetic polynucleotide

SEQ ID NO: 12 - DNA, 418 bp, Artificial Sequence
  Description of Artificial Sequence Synthetic polynucleotide
ttggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctgg

SEQ ID NO: 13 - DNA, 45 bp, Artificial Sequence
  Description of Artificial Sequence Synthetic oligonucleotide
ttggcagaactacacaccagggccagggatcagatatccactgac

SEQ ID NO: 14 - DNA, 190 bp, Artificial Sequence; identical to SEQ ID NO: 9
  Description of Artificial Sequence Synthetic polynucleotide

SEQ ID NO: 15 - DNA, 228 bp, Artificial Sequence
  Description of Artificial Sequence Synthetic polynucleotide
ttggcagaactacacaccagggccagggatcagatatccactgactactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctgg

SEQ ID NO: 16 - DNA, 34 bp, Human immunodeficiency virus 1
agggatcagatatccactgacctttggatggtgc

SEQ ID NO: 17 - DNA, 34 bp, Human immunodeficiency virus 1; identical to SEQ ID NO: 16

SEQ ID NO: 18 - DNA, 34 bp, Human immunodeficiency virus 1
aaaggatagatgtaaaagacaccaaggaagcctt

SEQ ID NO: 19 - DNA, 34 bp, Human immunodeficiency virus 1; identical to SEQ ID NO: 18

SEQ ID NO: 20 - DNA, 1400 bp, Human immunodeficiency virus 1
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagac

SEQ ID NO: 21 - DNA, 1400 bp, Human immunodeficiency virus 1; identical to SEQ ID NO: 20

SEQ ID NO: 22 - DNA, 312 bp, Artificial Sequence
  Description of Artificial Sequence Synthetic polynucleotide
ctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagc

(19) United States
(12) Patent Application Publication
(10) Pub. No.: US 2018/0073019 A1
(43) Pub. Date: Mar. 15, 2018

(54) TAT-INDUCED CRISPR/ENDONUCLEASE-BASED GENE EDITING

(71) Applicant: Temple University - of the Commonwealth System of Higher Education, Philadelphia, PA (US)

(72) Inventor: Kamel Khalili, Bala Cynwyd, PA (US)

(21) Appl. No.: 15/559,902

(22) PCT Filed: Mar. 18, 2016
(86) PCT No.: PCT/US2016/023170

§ 371 Date: Sep. 20, 2017

Related U.S. Application Data
(60) Provisional application No. 62/136,080, filed on Mar. 20, 2015.

(51) Int. Cl. C12N 15/11; C12N 9/22; A61K 48/00; C12N 15/86; A61K 45/06; C12N 7/00

(52) U.S. Cl. CPC C12N 15/111; C12N 9/22; C12N 15/11; A61K 48/005; C12N 15/86; A61K 45/06; C12N 2740/16062; C12N 7/00; C12N 2310/3519; C12N 2310/20; C12N 2830/60; C12N 2800/22; A61K 48/0075

(57) ABSTRACT

Compositions and methods are provided for Tat-inducible expression of a CRISPR-associated endonuclease by a truncated HIV LTR promoter containing at least a core region and a TAR region of a HIV LTR promoter. The compositions may be used as a therapeutic treatment for the treatment and/or prevention of HIV.

### FIELD OF THE INVENTION

The present invention relates to compositions that specifically cleave target sequences in retroviruses, for example human immunodeficiency virus (HIV). Such compositions, which can include nucleic acids encoding a Clustered Regularly Interspaced Short Palindromic Repeat (CRISPR) associated endonuclease and a guide RNA sequence complementary to a target sequence in a human immunodeficiency virus, can be administered to a subject having or at risk for contracting an HIV infection.

### BACKGROUND OF THE INVENTION

Since the discovery of HIV-1, AIDS remains a major public health problem affecting millions of people worldwide. AIDS remains incurable due to the permanent integration of HIV-1 into the host genome. Current therapy (highly active antiretroviral therapy or HAART) for controlling HIV-1 infection and impeding AIDS development profoundly reduces viral replication in cells that support HIV-1 infection and reduces plasma viremia to a minimal level. But HAART fails to suppress low level viral genome expression and replication in tissues and fails to target latently-infected cells, for example, resting memory T cells, brain macrophages, microglia, and astrocytes, gut-associated lymphoid cells, that serve as a reservoir for HIV-1. Persistent HIV-1 infection is also linked to comorbidities including heart and renal diseases, osteopenia, and neurological disorders. There is a continuing need for curative therapeutic strategies that target persistent viral reservoirs.

The HIV-1 genome is about 9.8 kb in length, including two viral long-terminal repeats located at both ends when integrated into the host genome. The genome also includes genes that encode for the structural proteins Gag, Pol, and Env, regulatory proteins (Tat and Rev), and accessory proteins Vpu, Vpr, Vif, and Nef. The HIV-1 transactivator of transcription (Tat) is a multifunctional protein that has been proposed to contribute to several pathological consequences of HIV-1 infection. Tat not only plays an important role in viral transcription and replication, it is also capable of inducing the expression of a variety of cellular genes as well as acting as a neurotoxic protein. Tat protein is secreted by HIV-1-infected cells and acts by diffusing through the cell membrane. It may act as a secreted, soluble neurotoxin and induces HIV-1-infected macrophages and microglia to release neurotoxic substances. Tat transcription is driven by the HIV-1 LTR promoter and is required for overall viral replication of HIV.

The clinical course of HIV infection can vary according to a number of factors, including the subject's genetic background, age, general health, nutrition, treatment received, and the HIV subtype. In general, most individuals develop flu-like symptoms within a few weeks or months of infection. The symptoms can include fever, headache, muscle aches, rash, chills, sore throat, mouth or genital ulcers, swollen lymph glands, joint pain, night sweats, and diarrhea. The intensity of the symptoms can vary from mild to severe depending upon the individual. During the acute phase, the HIV viral particles are attracted to and enter cells expressing the appropriate CD4 receptor molecules. Once the virus has entered the host cell, the HIV encoded reverse transcriptase generates a proviral DNA copy of the HIV RNA and the proviral DNA becomes integrated into the host cell genomic DNA. It is this HIV provirus that is replicated by the host cell, resulting in the release of new HIV virions which can then infect other cells.

The primary HIV infection subsides within a few weeks to a few months, and is typically followed by a long clinical “latent” period which may last for up to 10 years. The latent period is also referred to as asymptomatic HIV infection or chronic HIV infection. The subject's CD4 lymphocyte numbers rebound, but not to pre-infection levels and most subjects undergo seroconversion, that is, they have detectable levels of anti-HIV antibody in their blood, within 2 to 4 weeks of infection. During this latent period, there can be no detectable viral replication in peripheral blood mononuclear cells and little or no culturable virus in peripheral blood. During the latent period, also referred to as the clinical latency stage, people who are infected with HIV may experience no HIV-related symptoms, or only mild ones. But, the HIV virus continues to reproduce at very low levels. In subjects who have been treated with anti-retroviral therapies, this latent period may extend for several decades or more. However, subjects at this stage are still able to transmit HIV to others even if they are receiving antiretroviral therapy, although antiretroviral therapy reduces the risk of transmission.

CRISPRs (clustered regularly interspaced short palindromic repeats) are DNA loci containing short repetitions of base sequences. Each repetition is followed by short segments of “spacer DNA” from previous exposures to a virus. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms.

The CRISPR/Cas system has been used for gene editing (by adding, disrupting or changing the sequence of specific genes) and gene regulation in various organisms. By delivering the Cas9 protein and appropriate guide RNAs into a cell, the organism's genome can be cut at any desired location. Successful therapeutic gene editing using CRISPR/Cas9 system requires efficient and specific delivery and expression of Cas9 enzyme and guide RNAs in target cells. This is particularly challenging when the frequency of recipient cells in a tissue or cell population is low, such as in the case of certain virus-infected cells.

### SUMMARY

Provided herein is a method of inactivating a human immunodeficiency virus (HIV) in a mammalian cell in vivo or in vitro. The method includes exposing the mammalian cell to a composition that includes an isolated nucleic acid sequence encoding a clustered regularly interspaced short palindromic repeats (CRISPR)-associated endonuclease operably linked to a truncated HIV LTR promoter containing at least a core region and a TAR region of a HIV LTR promoter.

In specific embodiments, the CRISPR-associated endonuclease is Cas9. The CRISPR-associated endonuclease may be optimized for expression in a human cell. The exposing of the mammalian cell to the composition may include contacting the cell. The mammalian cell may be a latently infected cell including, but not limited to, a CD4⁺ T cell, a macrophage, a monocyte, a gut-associated lymphoid cell, a microglial cell, and an astrocyte. The mammalian cell may include a cultured cell from a subject having a HIV infection, a tissue explant, and/or a cell line. The inactivating of HIV may be performed in vivo or ex vivo.

In specific embodiments, the isolated nucleic acid may additionally encode one or more guide RNAs that are complementary to a target nucleic acid sequence in HIV. The target nucleic acid sequence in HIV may refer to a sequence within a coding and/or noncoding region and/or the long terminal repeat of HIV. The non-coding region may include a long terminal repeat of HIV. A sequence within the long terminal repeat of HIV may include a sequence within U3, R, or U5 regions that excludes any sequence of the truncated HIV LTR promoter. The composition may include a sequence that encodes a nuclear localization signal. The composition may additionally include a sequence encoding a transactivating small RNA (tracrRNA) and the tracrRNA may be fused to a sequence encoding a guide RNA. The composition may also include an enhancer region of the HIV LTR promoter.

In specific embodiments, the composition may be operably linked to an expression vector. The expression vector may be a lentiviral vector, an adenoviral vector, and an adeno-associated virus vector.

Provided herein is an isolated nucleic acid sequence that includes a sequence encoding a CRISPR-associated endonuclease operably linked to a truncated HIV LTR promoter containing at least a core region and a TAR region of a HIV LTR promoter.

In specific embodiments, the CRISPR-associated endonuclease is Cas9. The CRISPR-associated endonuclease may be optimized for expression in a human cell.

In specific embodiments, the sequence may additionally encode one or more guide RNAs that are complementary to a target nucleic acid sequence in HIV. The target nucleic acid sequence in HIV may refer to a sequence within a coding and/or noncoding region and/or the long terminal repeat of HIV. The long terminal repeat of HIV may include a sequence within the U3, R, or U5 regions that excludes any sequence of the truncated HIV LTR promoter. The isolated nucleic acid sequence may also encode a nuclear localization signal and/or a transactivating small RNA (tracrRNA). The tracrRNA may be fused to a sequence encoding a guide RNA. The isolated nucleic acid sequence may also include an enhancer region of the HIV LTR promoter.

In specific embodiments, the isolated nucleic acid sequence may be operably linked to an expression vector. The expression vector may refer to a lentiviral vector, an adenoviral vector, and an adeno-associated virus vector.

Provided herein is a pharmaceutical composition that includes a sequence encoding a CRISPR-associated endonuclease operably linked to a truncated HIV LTR promoter containing at least a core region and a TAR region of a HIV LTR promoter. The pharmaceutical composition may also include a pharmaceutically acceptable carrier including, but not limited to, a lipid-based or polymer-based colloid. The colloid may be a liposome, a hydrogel, a microparticle, a nanoparticle, or a block copolymer micelle. In specific embodiments, the CRISPR-associated endonuclease is Cas9. The CRISPR-associated endonuclease may be optimized for expression in a human cell.

In specific embodiments, the pharmaceutical composition may be formulated for topical application and/or contained within a condom.

In specific embodiments, the sequence may additionally encode one or more guide RNAs that are complementary to a target nucleic acid sequence in HIV. The target nucleic acid sequence in HIV may refer to a sequence within a coding and/or noncoding region and/or the long terminal repeat of HIV. The sequence within the long terminal repeat of HIV may include a sequence within the U3, R, or U5 regions that excludes any sequence of the truncated HIV LTR promoter. The sequence may encode a nuclear localization signal. The pharmaceutical composition may additionally include a sequence encoding a tracrRNA and the tracrRNA may be fused to a sequence encoding a guide RNA. The sequence may also encode an enhancer region of the HIV LTR promoter.

In specific embodiments, the sequence provided by the pharmaceutical composition may be operably linked to an expression vector. The expression vector may be a lentiviral vector, an adenoviral vector, and an adeno-associated virus vector.

Provided herein is a method of treating a subject having a HIV infection. The method includes administering to the subject a therapeutically effective amount of a pharmaceutical composition that includes a sequence encoding a CRISPR-associated endonuclease operably linked to a truncated HIV LTR promoter containing at least a core region and a TAR region of a HIV LTR promoter. The HIV infection being treated may be a latent infection. The method may further include identifying a subject having a HIV infection.

In specific embodiments, the CRISPR-associated endonuclease is Cas9. The CRISPR-associated endonuclease may be optimized for expression in a human cell.

In specific embodiments, the sequence may additionally encode one or more guide RNAs that are complementary to a target nucleic acid sequence in HIV. In some instances, the sequence may encode an enhancer region of the HIV LTR promoter.

In specific embodiments, an anti-retroviral agent may be administered. The anti-retroviral agent may include, but is not limited to, non-nucleoside reverse transcriptase inhibitors, protease inhibitors, and entry inhibitors. The anti-retroviral agent may include highly active antiretroviral therapy. The pharmaceutical composition may be administered topically or parenterally.

In specific embodiments, the pharmaceutical composition may be operably linked to an expression vector. The expression vector may be a lentiviral vector, an adenoviral vector, and an adeno-associated virus vector.

Provided herein is a method of reducing risk of a HIV infection in a subject at risk for the HIV infection. The method may include administering to the subject a therapeutically effective amount of a pharmaceutical composition comprising a sequence encoding a CRISPR-associated endonuclease operably linked to a truncated HIV LTR promoter containing at least a core region and a TAR region of a HIV LTR promoter. In an embodiment, the subject is sexually active, a health care worker, and/or a first responder.

In specific embodiments, the CRISPR-associated endonuclease may be Cas9. The CRISPR-associated endonuclease may be optimized for expression in a human cell. In some instances, the pharmaceutical composition may be operably linked to an expression vector. The expression vector may be, without limitation, a lentiviral vector, an adenoviral vector, and an adeno-associated virus vector. In an embodiment, the sequence may also encode an enhancer region of the HIV LTR promoter.

Provided herein is a method of reducing risk of transmission of a HIV infection from a HIV-infected gestating or lactating mother to her offspring. The method includes administering to the subject a therapeutically effective amount of a pharmaceutical composition that includes a sequence encoding a CRISPR-associated endonuclease operably linked to a truncated HIV LTR promoter containing at least a core region and a TAR region of a HIV LTR promoter. In an embodiment, the pharmaceutical composition is administered during one or more of: prenatally, perinatally, and postnatally.

In specific embodiments, an anti-retroviral agent may be administered. The anti-retroviral agent may be, without limitation, non-nucleoside reverse transcriptase inhibitors, protease inhibitors, and entry inhibitors. The anti-retroviral agent may be highly active antiretroviral therapy. In an embodiment, a therapeutically effective amount of the composition may be administered to the offspring. In an embodiment, the sequence may also encode an enhancer region of the HIV LTR promoter.

Provided herein is a method of administering a pharmaceutical composition to prevent infection by a HIV in an uninfected subject. The method may include administering to the uninfected subject a therapeutically effective amount of the pharmaceutical composition that includes a sequence encoding a CRISPR-associated endonuclease operably linked to a truncated HIV LTR promoter containing at least a core region of a HIV LTR promoter and a TAR region of a HIV LTR promoter.

Provided herein is a kit that includes a measured amount of a composition that includes an isolated nucleic acid sequence that includes a sequence encoding a CRISPR-associated endonuclease operably linked to a truncated HIV LTR promoter containing at least a core region and a TAR region of a HIV LTR promoter, or a vector encoding the isolated nucleic acid, and one or more items of: packaging material, a package insert including instructions for use, a sterile fluid, a syringe, and a sterile container.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

### BRIEF DESCRIPTION OF THE DRAWINGS

FIG. 1A is a schematic representation of full length HIV-1 LTR (LTR(−454/+66)), and created LTR truncation variants LTR −120/+66, LTR −80/+66 and LTR −38/+66. The LTR elements contained in each variant are apparent from the figure.

FIG. 1B is an agarose gel electrophoresis image of PCR-amplified LTR sequences of full length HIV-1 LTR and the variants of FIG. 1A. Lane 1: full length HIV-1 LTR (pLTR(−454/+66)). Lane 2: pLTR (−120/+66). Lane 3: pLTR (−80/+66. Lane 4: pLTR (−38/+66).

FIG. 2 is a diagram of a Cas9 promoter replacement procedure according to the present invention. pX260-U6-DR-BB-DR-Cbh-NLS-hSpCas9-NLS-H1-shorttracr-PGK-puro plasmid (Addgene #42229) (marked as “CBh-Cas9”), was used as a Cas9 gene source/template. The original CBh promoter in the reference plasmid was removed by restriction enzyme digestion with the enzymes indicated in the figure and replaced with different HIV-1 LTR promoter variants (marked collectively as “LTR-Cas9”).

FIG. 3A is a Western blot of Cas9, Tat and α-tubulin expression in U87 MG cells co-transfected with different amounts of plasmids expressing FLAG-labeled Cas9 under control of full length HIV-1 LTR (pLTR(−454/+66)-FLAG-Cas9) (10, 50 and 250 ng), with or without Tat expressing plasmid (pCMV-Tat86, 250 ng). Lane 1: pLTR(−454/+66)-Cas9 250 ng, pCMV 1000 ng. Lane 2: pLTR(−454/+66)-Cas9 50 ng, pCMV 1200 ng. Lane 3: pLTR(−454/+66)-Cas9 10 ng, pCMV 1240 ng. Lane 4: pLTR(−454/+66)-Cas9 250 ng, pCMV 750 ng, pCMV-Tat86 250 ng. Lane 5: pLTR(−454/+66)-Cas9 50 ng, pCMV 950 ng, pCMV-Tat86 250 ng. Lane 6: pLTR(−454/+66)-Cas9 10 ng, pCMV 990 ng, pCMV-Tat86 250 ng.

FIG. 3B comprise graphs of the intensity of the bands corresponding to Cas9 and normalized to α-tubulin expression in the Western blot of FIG. 3A. The top panel show the Western blot image quantification of the Cas9 levels normalized to α-tubulin levels, with or without Tat. The bottom panel show the Western blot image quantification of the +Tat/no Tat ratio.

FIG. 4A is a Western blot of Cas9, Tat and α-tubulin expression in U87 MG cells transfected with different amounts of plasmids (5 ng or 50 ng) expressing FLAG-labeled Cas9 under control of the HIV-1 truncated LTR variant pLTR(−120/+66)-FLAG-Cas9 or the HIV-1 LTR variant pLTR(−80/+66)-FLAG-Cas9, with or without Tat expressing plasmid (pCMV-Tat86, 250 ng). Lane 1: pLTR(−120/+66)-Cas9 5 ng, pCMV 1245 ng. Lane 2: pLTR(−120/+66)-Cas9 5 ng, pCMV 1245 ng, +rTat protein 2.5 μg/ml. Lane 3: pLTR(−120/+66)-Cas9 5 ng, pCMV 995 ng, pCMV-Tat86 250 ng. Lane 4: pLTR(−120/+66)-Cas9 50 ng, pCMV 1200 ng. Lane 5: pLTR(−120/+66)-Cas9 50 ng, pCMV 1200 ng, +rTat protein 2.5 μg/ml. Lane 6: pLTR(−120/+66)-Cas9 50 ng, pCMV 950 ng, pCMV-Tat86 250 ng. Lane 7: pLTR(−80/+66)-Cas9 5 ng, pCMV 1245 ng. Lane 8: pLTR(−80/+66)-Cas9 5 ng, pCMV 1245 ng, +rTat protein 2.5 μg/ml. Lane 9: pLTR(−80/+66)-Cas9 5 ng, pCMV 995 ng, pCMV-Tat86 250 ng. Lane 10: pLTR(−80/+66)-Cas9 50 ng, pCMV 1200 ng. Lane 11: pLTR(−80/+66)-Cas9 50 ng, pCMV 1200 ng, +rTat protein 2.5 μg/ml. Lane 12: pLTR(−80/+66)-Cas9 50 ng, pCMV 950 ng, pCMV-Tat86 250 ng.

FIG. 4B comprise graphs of the intensity of the bands corresponding to Cas9 normalized to α-tubulin expression in the Western blot of FIG. 4A. The top panel show the Western blot image quantification of the Cas9 levels normalized to α-tubulin levels, with no Tat, with rTAT or with transfected Tat. The bottom panel show the Western blot image quantification of the +Tat(transfected)/no Tat ratio.

FIGS. 5A-5E depict the expression of Cas9 by the HIV-1 LTR promoter is stimulated by Tat leading to cleavage of the viral promoter in the presence of gRNA. FIG. 5A: Schematic presentation of the full-length HIV-1 LTR and the various regulatory motifs within the enhancer and core regions, and the partial Gag gene. The extent of LTR deletion mutants that are created for expression of Cas9 is depicted. The positions of the gRNA target sequence and their distance from each other is shown. FIG. 5B: Co-transfection of TZMb1 cells with pX260-LTR-Cas9 containing the full-length LTR (−454/+66) or its various mutants (−120/+66 or −80/+66) along with a plasmid expressing Tat (pCMV-Tat) increased the level of Tat production as tested by Western blot (top panel). Expression of housekeeping α-tubulin (middle panel) and Tat (bottom panel) are shown. FIG. 5C: Infection of TZMb1 cells with adenovirus expressing Tat, at two different multiplicities of infection (MOI) followed by lentivirus mediated expression of Cas9 by the LTR_{-80/+66} promoter and gRNAs A/B by the U6 promoter led to cleavage of the integrated HIV-1 LTR promoter DNA sequence and the appearance of a 205 bp DNA fragment in the TZMb1 cells (as tested by PCR and DNA gel analysis). FIG. 5D: SDS-PAGE illustrating the level of Cas9, β-tubulin and Tat protein expressed in TZMb1 cells as described in FIG. 5C. FIG. 5E: Luciferase assay illustrating transcriptional activity of the integrated HIV-1 LTR in TZMb1 cells after various treatments as described in FIG. 5 C.

FIGS. 6A-6C show that HIV-1 infection stimulates cleavage of integrated viral DNA upon induction of Cas9. The LTR_{-80/+66}-Cas9 reporter TZMb1 cell line transduced with lentivirus expressing gRNA A/B (LV-gRNA A/B) or control (empty LV) was infected with three different MOI of HIV-1_{JRFL} or HIV-1_{SF162}, and after 48 hours, cells were harvested and protein expression was determined by Western blot (FIG. 6A), the level of integrated HIV-1 LTR cleavage upon induction of Cas9 after viral infection was detected by PCR/DNA gene analysis (FIG. 6B) and transcriptional activity of the integrated HIV-1 promoter was evaluated by luciferase reporter assay (FIG. 6C).

FIGS. 7A-7C show that Tat stimulation of Cas9 cleaves integrated HIV-1 DNA in T-cells encompassing the HIV-1 reporter at a latent stage. CD4⁺ Jurkat T-cells, 2D10 cells, containing LTR_{-80/+66}-Cas9 gene were transduced with control (empty LV) or LV-gRNA A/B followed by transfection with pCMV or pCMV-Tat plasmids. After 48 hours, the level of various proteins, as depicted, was determined by Western blot (FIG. 7A). The genomic DNA for assessing the state of the integrated HIV-1 DNA was determined by LTR specific PCR and the excision efficiency was determined as a percentage of ratios between truncated vs. full-length amplicon (FIG. 7B). The level of integrated viral promoter reactivation after cleavage was assessed by flow cytometry and the representative scatter plots are shown (FIG. 7C). Red positive, propidium iodide stained, and dead cells were excluded from the analysis.

FIGS. 8A-8C show that treatment of cells with latency reversing drugs induces Cas9 expression and cleavage of integrated viral DNA in Jurkat 2D10 cells. 2D10 cells expressing LTR_{-80/+66}-Cas9 were treated with control (empty) or lentivirus expressing gRNAs A/B and 24 hours later they were treated with PMA (P), TSA (T) or both (P/T) for 16 hours. Protein studies for the expression of Cas9-Flag, α-tubulin and GFP (indicative of the integrated HIV-1 genome) was determined by Western blot (FIG. 8A). Genomic DNA for the detection of the level of excision within the integrated LTR DNA by Cas9 and gRNA A/B was assessed by PCR and the excision efficiency was determined as described in FIG. 7A-7C legend (FIG. 8B). GFP reporter assay, by flow cytometry, and representative scatter plot is shown (FIG. 8C).

FIG. 9 is a schematic representation of negative feedback regulation of HIV-1 by CRISPR/Cas9. At the early stage of (reactivation), basal transcription of the viral genome allows production of Tat protein \textcircled{1}. Upon association of TAT with the budge sequence of the viral transcript \textcircled{2} and recruitment of several cellular protein to associate with the loop of TAR and other transcription factors at RNA poly II in close proximity of the transcription start site, transcription of viral RNA is highly stimulated at the initiation and more importantly, elongation \textcircled{3}. The basal product upon viral activation also stimulates the minimum viral promoter, ltr, driving the Cas9 gene \textcircled{3}. The newly synthesized Cas9 upon association with various HIV-1 specific gRNAs, cleaves the viral genome and permanently inactivates the LTR and shuts down HIV-1 gene expression and replication. In the absence of Tat, ltr-Cas9 becomes silent. Expression of Cas9 can continue only in the presence of Tat.

FIG. 10 shows the position and nucleotide sequences of gRNA A/B targets within the LTR (highlighted in green, PAM in red) and LTR specific primers used in PCR on TZMb1 genomic DNA (highlighted in blue) in the reference HIV-1 NL4-3 genome. Sequences and sizes of LTR specific PCR products (full-length and truncated) and predicted edited fragment (SEQ ID NOS: 6-10).

FIG. 11 shows a representative agarose gel analyzing LTR specific PCR reactions used for quantification of Cas9/gRNA mediated LTR excision efficiency in experiments using the Jurkat 2D10 reporter cell line from FIGS. 7A-7C and 8A-8C.

FIG. 12 shows the position and nucleotide composition of LTR gRNA A/B targets (highlighted in green, PAM in red) and LTR specific primers used to analyze excision by PCR in Jurkat 2D10 cells (highlighted in blue) in the reference HIV-1 NL4-3 genome. Nucleotide sequences and sizes of amplicons (full-length and truncated LTR DNA) and predicted excised DNA fragment are shown (SEQ ID NOS: 11-21).

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

As used herein, the terms “nucleic acid sequence”, “polynucleotide,” are used interchangeably throughout the specification and include complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The term “target nucleic acid” refers to a nucleic acid (often derived from a biological sample), to which the oligonucleotide is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” means a DNA sequence recognized by the synthetic machinery of the cell, or introduced synthetic machinery, required to initiate the specific transcription of a polynucleotide sequence. A “minimal” promoter or “truncated” promoter or “functional fragment” of a promoter includes all essential elements of a promoter for transcriptional activation of, for example, a nucleic acid sequence operably linked or under control of the minimal promoter. In one embodiment, a truncated HIV long terminal repeat (LTR) promoter comprises at least a core region, a trans activation response element (TAR) or combinations thereof, of a HIV LTR promoter.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

The term “percent sequence identity” refers to the degree of identity between any given query sequence and a subject sequence.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

As used herein, the term “kit” refers to any delivery system for delivering materials. Inclusive of the term “kits” are kits for both research and clinical applications. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides or liposomes. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

### DETAILED DESCRIPTION

Soon after infection with HIV-1, the viral genome becomes integrated into the host chromosome and is rapidly expressed in CD4⁺ T-cells. HIV-1 replication leads to drastic depletion of CD4⁺ T-cells. Often, after the acute phase of infection, the virus enters a new phase called latency, where the integrated proviral DNA continues to be expressed and viral replication proceeds at very low levels. Under these circumstances, the weakened immune system caused by persistent viral replication progresses to AIDS and the development of a broad range of opportunistic infections that eventually lead to death within three years if untreated. At the molecular level, expression of the viral genome and its replication both at the acute and chronic states is controlled by the viral promoter that spans 450 nucleotides of the 5′ long terminal region (LTR). Cooperativity occurs between a series of cellular transcriptional factors that recognize DNA sequences within the U3 region of the 5′-LTR and the HIV-1 immediate early transcription activator, Tat, which interacts with the TAR RNA sequence positioned within the leader region of the viral transcript. These interactions are required for the robust initiation and efficient elongation of transcription from integrated copies of the viral DNA. While the current anti-retroviral drugs have been effective in suppressing viral infection cycles, they have yet to contain any components that inhibit viral gene expression at the transcriptional level, supporting the notion that the integrated copies of the virus may continue to express the viral genome, albeit at very low levels, in HIV-1 positive patients under active antiretroviral therapy (ART). Indeed, expression of viral genes drastically elevates upon cessation of ART and allows production of viral early regulatory proteins such as Tat to orchestrate productive replication of the viral genome.

Accordingly, embodiments of the invention are directed to compositions for conditional activation of the CRISPR/Cas at the early stage of reactivation. These compositions completely and permanently ablate virus replication prior to productive viral replication by removing a segment of the viral gene spanning the viral promoter and/or the viral coding sequence. In embodiments, a composition comprises a nucleic acid sequence encoding a clustered regularly interspaced short palindromic repeats (CRISPR)-associated endonuclease (CRISPR/Cas) operably linked to a truncated functional viral promoter whereby the truncated viral promoter is under control of an immediate early transcriptional activator, thereby conditionally activating CRISPR/Cas at an early stage of viral replication. The isolated nucleic acid further comprises at least one guide RNA that is complementary to a target nucleic acid sequence in the virus. The CRISPR/Cas excises a segment of a viral genome, for example, the segment spanning a viral promoter and/or viral coding sequence. In these embodiments, the composition is tailored to excise any virus. In certain embodiments, the virus is a retrovirus.

A viral genome, e.g. HIV integrated into an infected host cell's genome may be eliminated from such HIV infected cells utilizing an RNA-guided clustered regularly interspaced short palindromic repeat (CRISPR)-associated endonuclease such as a Cas9. Successful therapeutic gene editing using CRISPR/Cas9 enzyme and guide RNA requires efficient and specific delivery and expression of Cas9 enzyme and guide RNAs in target cells. This is difficult when the frequency of recipient cells in a tissue or population of cells is low, such as HIV infected cells in patients on highly active antiretroviral therapy (HAART).

According to the present invention, a CRISPR-associated endonuclease such as a Cas9 is placed under the control of a truncated Tat-responsive HIV LTR promoter. The endonuclease expression is thereby activated in cells containing the Tat protein. As demonstrated herein, both exogenously provided (e.g., by transfection) and endogenously produced (e.g., by reactivation of latent virus) Tat can activate (CRISPR)-associated endonuclease (e.g., Cas9) expression in cells lines when expression of the endonuclease is placed under the control of the truncated Tat-responsive HIV LTR promoter. In the studies presented further detail in the examples section, the compositions allow for the conditional activation of the CRISPR/Cas9 at the early stage of viral reactivation by the HIV-1 transcriptional activator, Tat. This strategy completely and permanently ablates virus replication prior to productive viral replication by removing an entire viral genome or a segment of the viral gene spanning the viral promoter and/or the viral coding sequence.

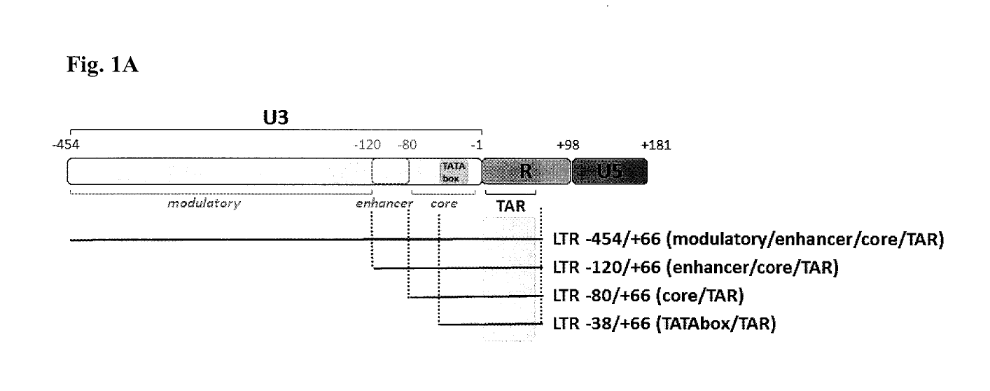

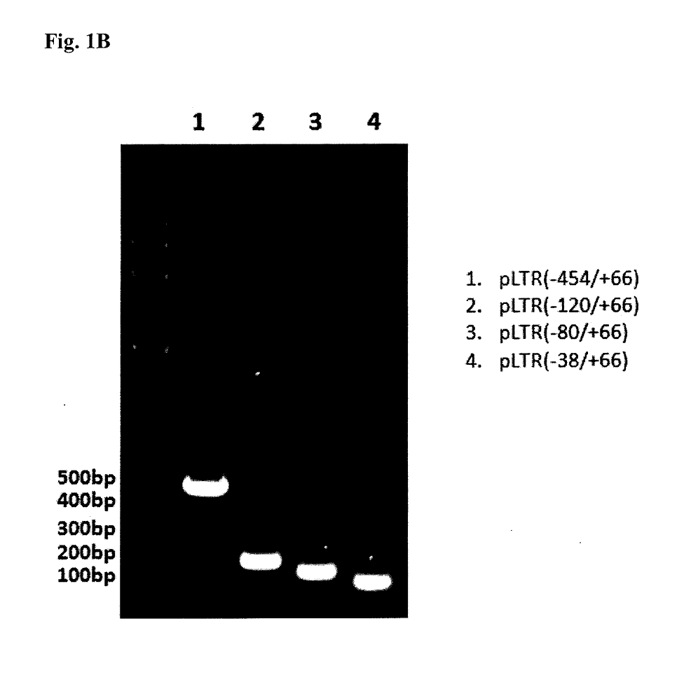

FIG. 1A shows a schematic representation of the HIV LTR. It is approximately 640 bp in length. HIV-1 LTR is divided into U3, R, and U5 regions. Transcription of the HIV-1 genome is controlled by a series of cis-acting regulatory motifs spanning the long-terminal region of the viral genome at the 5′ end. The U3 region of the viral promoter occupies −1 to −454 nucleotides, with respect to the transcription start site at +1 and has three sub-regions: modulatory, enhancer, and core. The enhancer contains the NF-κB binding site (−127 to −80). The core domain comprises the GC-rich and TATA box (−80 to +1). The R region (+1 to +98) of the LTR comprises TAR, a region for which the expressed RNA forms a stem-loop structure and provides a binding site for the viral transactivator (Krebs et al, Lentiviral LTR-directed expression, sequence variation, disease pathogenesis. Los Alamos National Laboratory HIV Sequence: Compendium, pp. 29-70.2002).

The LTRs contain all of the required signals for gene expression and are involved in the integration of a provirus into the genome of a host cell. For example, the core promoter, an enhancer, and a modulatory region are found within U3 while the TAR is found within R as shown in FIG. 1A. TAR, the binding site for Tat protein and for cellular proteins, consists of approximately the first 45 nucleotides of the viral mRNAs in HIV-1 forms a hairpin stem-loop structure. In HIV-1, the U5 region includes several sub-regions, for example, including Poly A which is involved in dimerization and genome packaging, PBS or primer binding site, Psi or the packaging signal, and DIS or dimer initiation site.

According to the present invention, a composition is provided comprising an isolated nucleic acid encoding a CRISPR-associated endonuclease operably linked to a truncated HIV LTR promoter containing at least the core region and the TAR (transactivation response element) region of HIV LTR promoter. A truncated HIV LTR promoter refers to an operative functional promoter containing less than the full length HIV LTR promoter. Preferably, the truncated promoter contains a core region and a TAR region without all or substantially all of the modulatory and/or enhancer regions. In another embodiment, the truncated HIV LTR promoter contains the core region, the TAR region, and all or substantially all of the enhancer region, but does not contain any of the modulatory region. The truncated HIV LTR promoter is responsive to Tat protein. That is, Tat can activate the expression of the CRISPR-associated endonuclease, such as Cas9, operably linked to the truncated HIV LTR promoter. The disclosed composition may be utilized to inactivate HIV in a mammalian cell, treat a subject having a HIV infection, reduce the risk of HIV infection in a subject at risk for infection, and/or reduce the risk of transmission of HIV from a HIV-infected mother to her offspring. The therapeutic methods disclosed herein may be carried out in connection with other antiretroviral therapies such as HAART. The composition may be included as a part of a kit for diagnostic, research, and/or therapeutic applications.

Anti-retroviral therapy does not suppress low levels of viral genome expression nor does it efficiently target latently infected cells such as resting memory T cells, monocytes, macrophages, microglia, astrocytes, and gut associated lymphoid cells as described earlier. However, the methods and compositions disclosed herein are generally useful for treatment of HIV infected subjects at any stage of infection, or to an uninfected subject who is at risk for HIV infection. In particular, the disclosed methods and compositions are useful for HIV infected subjects who are in the latent period of the infection. Moreover, when a guide RNA is associated with the CRISPR-associated endonuclease operably linked to a truncated, Tat-responsive HIV LTR promoter, as disclosed herein, the HIV genome may be excised from the host cell and eliminated.

Several advantages may be realized with the compositions containing a sequence encoding CRISPR-associated endonuclease operably linked to a truncated HIV LTR promoter containing the core region and the TAR region of HIV LTR promoter. The potential risk of toxic effects caused by the continuous expression may be alleviated and/or eliminated by limiting the expression of the CRISPR-associated endonuclease to cells with HIV gene expression and/or replication. For example, the potential to induce toxicity due to the immunogenicity of the CRISPR-associated endonuclease may be mitigated because of the low and/or intermittent expression of the endonuclease according to the present invention, while at the same time eliminate or cause self-destruction of the HIV genome in infected individuals. In addition, the present invention may provide a prophylactic strategy for at risk individuals because persistent expression of the CRISPR-associated endonuclease is minimized. Thus, the CRISPR-associated endonuclease driven by a truncated, Tat-responsive HIV LTR promoter may be utilized to provide a safe treatment of HIV infected subjects, and to vaccinate uninfected individuals who may be at risk of infection.

In some embodiments the promoter comprises one or more mutations, deletions, insertions, variants, derivatives or combinations thereof. The promoter may also be chimeric, comprising one or chimeric compounds.

Placing the CRISPR-associated endonuclease under control of a truncated HIV LTR promoter, as described herein, is also advantageous because a smaller-sized nucleic acid may be more readily packaged into delivery mechanisms suitable for gene therapy (e.g., retroviruses). Promoter constructs that include the modulatory region, for example, may be less suitable for gene therapy due to their size and/or variable effects on transcription of CRISPR-associated endonuclease. Further, a composition including only the TATA box of the core region plus the full TAR region of the HIV LTR promoter is unable to adequately express Cas9 (data not shown). Compositions including the entire core region, the TAR region, and optionally the enhancer of the HIV-1 LTR promoter (see FIG. 1A) are able to drive Tat-induced expression of Cas9 in a dosage dependent manner.

The truncated HIV-1 LTR promoter may comprise a nucleic acid that includes the nucleotides of positions −80 to +66 of the HIV-1 LTR promoter. In an embodiment, the truncated HIV-1 LTR promoter may comprise a nucleic acid that includes positions −120 to +66 of the HIV-1 LTR promoter. Preferably, the truncated HIV-1 LTR promoter does not contain sequences from the modulatory region.

As disclosed herein, full length and truncated HIV-1 LTR promoter sequences were obtained by PCR using pNL4-3 HIV vector (NIH AIDS Reagent program #114) as a template and the primers shown in the table below:

| Primer name | Sequence | SEQ ID NO. |
|---|---|---|
| Kpn1-LTR | 5′-GGTACCTGGAAGGG | SEQ ID NO: 1 |
| (−454)-S | CTAATTTGG-3′ | |
| Kpn1-LTR | 5′-GGTACCTCGAGCTT | SEQ ID NO: 2 |
| (−120)-S | TCTACAAGG-3′ | |
| Xba1-LTR | 5′-TCTAGAGGAGGTGT | SEQ ID NO: 3 |
| (−80)-S | GGCCTGGGC-3′ | |
| Kpn1-LTR | 5′-GGTACCAGATGCTA | SEQ ID NO: 4 |
| (−38)-S | CATATAAGC-3′ | |
| LTR(+66)- | 5′-CCATGGTAAGCAGT | SEQ ID NO: 5 |
| Nco1-AS | GGGTTCC-3′ | |

The bolded nucleotides in the sequence column correspond to the cleavage site of the restriction enzyme bolded in the respective primer name column. Each primer was utilized to generate a different-sized segment of the HIV-1 LTR promoter sequence as shown in FIG. 1A. For example, LTR −454/+66 includes the entire U3 region and a portion of the R region. In contrast, LTR −80/+66 corresponds to the core region of U3 and the TAR region of R. The LTR −38/+66 nucleotide sequence was unable to adequately drive expression of Cas9 in response to Tat at a detectable level (data not shown).

The truncated HIV-1 LTR promoter of the present invention corresponds to a segment containing the core region of U3 and the TAR. The core region includes the TATA box and a GC rich region that may be a target for SP1. In some configurations, the truncated HIV-1 LTR promoter may include the enhancer at positions −120 to −80 as shown in FIG. 1A.

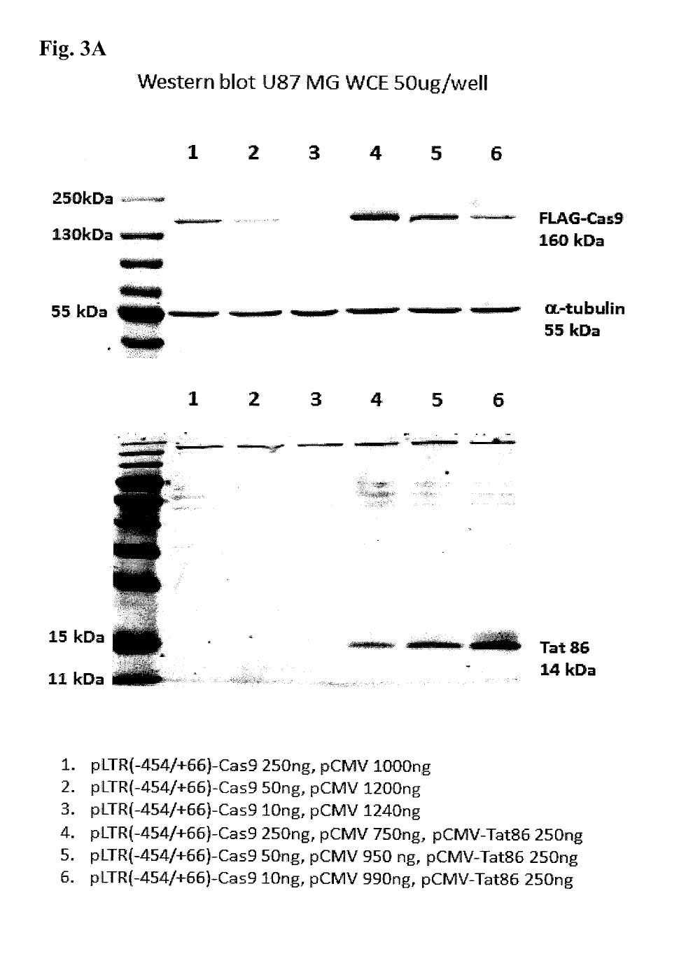

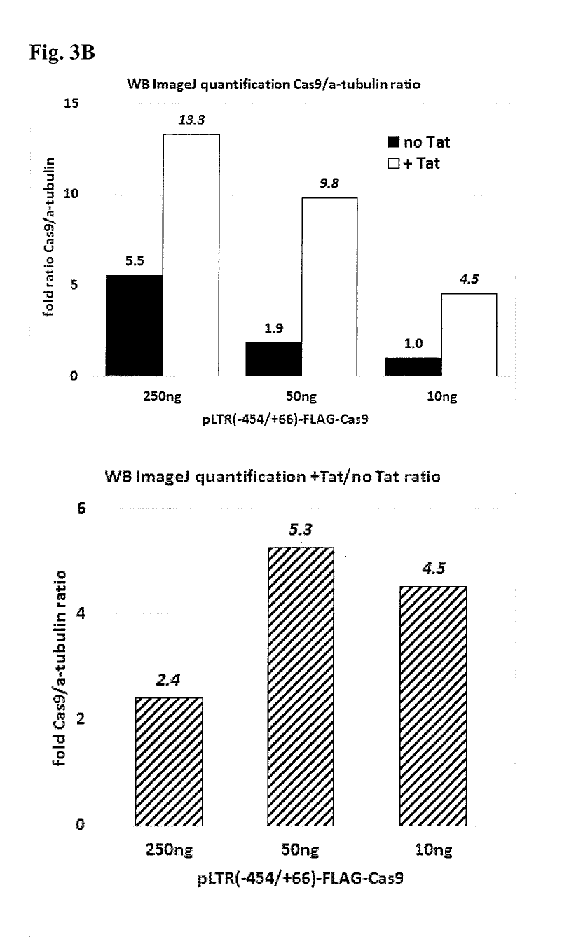

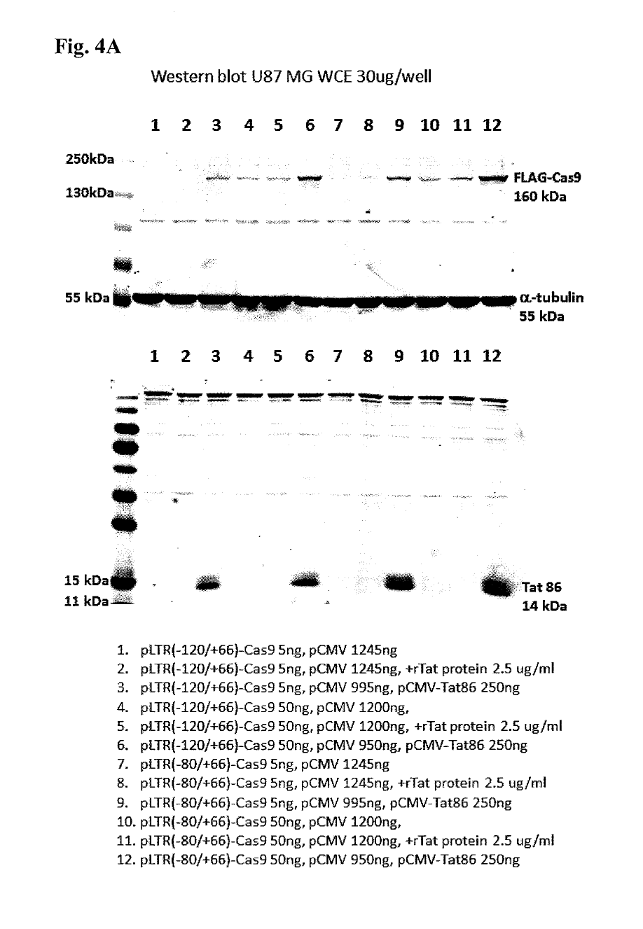

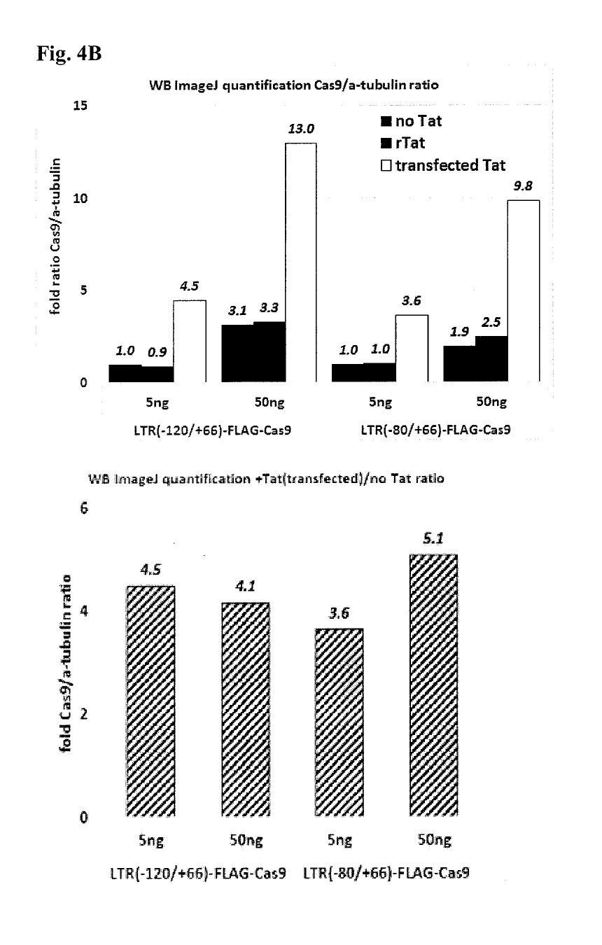

The truncated HIV-1 LTR promoter may be utilized to drive expression of a CRISPR-associated endonuclease such as Cas9. Such endonucleases are described in PCT international application No. PCT/US2014/053441 (WO2015/031775) filed on Aug. 29, 2014 and published on Mar. 5, 2015, the entire disclosure of which is incorporated herein by reference. As described above, the HIV genome integrates into a host genome of an individual infected with HIV. This integrated sequence is then replicated by the host. Even in the latent period, Tat may be produced by the cell. The compositions of the present invention eliminate and/or reduce the presence of the proviral polynucleotides in the host. Because the CRISPR-associated endonuclease is driven by a Tat-responsive promoter according to the present invention, any time Tat is present (e.g., produced by an infected cell), the endonuclease is produced and degrades the nascent polynucleotides. When the virus is not active, no endonuclease is produced. Thus avoided are potential toxic effects that continual expression of the endonuclease may exert on the cell and/or host. Moreover, the amount of endonuclease produced is proportional to the amount of Tat present as described below with respect to FIGS. 3 and 4.

In certain embodiments, an isolated nucleic acid sequence has at least a 50% sequence similarity to any one of SEQ ID NOS: 1 to 21.

In certain embodiments, an isolated nucleic acid sequence has at least a 70% sequence similarity to any one of SEQ ID NOS: 1 to 21.

In certain embodiments, an isolated nucleic acid sequence has at least a 75% sequence similarity to any one of SEQ ID NOS: 1 to 21.

In certain embodiments, an isolated nucleic acid sequence has at least an 85% sequence similarity to any one of SEQ ID NOS: 1 to 17 to about 95%, 96%, 97%, 98%, or 99% sequence similarity to any one of SEQ ID NOS: 1 to 21.

In certain embodiments, an isolated nucleic acid sequence comprises any one of SEQ ID NOS: 1 to 21 or combinations thereof.

The compositions disclosed herein may include nucleic acids encoding a CRISPR-associated endonuclease, such as Cas9. In some embodiments, one or more guide RNAs that are complementary to a target sequence of HIV may also be encoded. In bacteria, the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

The CRISPR-associated endonuclease can be a Cas9 nuclease. The Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficle, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Psuedomona aeruginosa, Escherichia coli, or other sequenced bacteria genomes and archaea, or other prokaryotic microogranisms may also be a source of the Cas9 sequence utilized in the embodiments disclosed herein.

The wild type Streptococcus pyogenes Cas9 sequence can be modified. The nucleic acid sequence can be codon optimized for efficient expression in mammalian cells, i.e., “humanized.” sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765 or Cas9 amino acid sequence of PX330 or PX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and Lcyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

The compositions and methods of the present invention may include a sequence encoding a guide RNA that is complementary to a target sequence in HIV. The genetic variability of HIV is reflected in the multiple groups and subtypes that have been described. A collection of HIV sequences is compiled in the Los Alamos HIV databases and compendiums (i.e., the sequence database web site is hitp://www.hiv.lani.gov). The methods and compositions of the invention can be applied to HIV from any of those various groups, subtypes, and circulating recombinant forms. These include for example, the HIV-1 major group (often referred to as Group M) and the minor groups, Groups N, 0, and P, as well as but not limited to, any of the following subtypes, A, B, C, D, F, G, H, J and K. or group (for example, but not limited to any of the following Groups, N, 0 and P) of HIV.

The guide RNA can be a sequence complimentary to a coding or a non-coding sequence (i.e., a target sequence). For example, the guide RNA can be a sequence that is complementary to a HIV long terminal repeat (LTR) region other than the portions that are utilized informing the truncated Tat-responsive promoter that is operably linked to the Cas9 gene. The guide RNA cannot target the sequence corresponding to the truncated Tat-responding HIV-1 LTR promoter as disclosed herein because it would result in degradation of the construct itself, thereby potentially removing the advantages rendered by the CRISPR-associated endonuclease driven by the truncated HIV LTR promoter. Thus, a guide RNA can include a sequence found within an HIV-1 U3, R, and/or U5 region reference sequence or consensus sequence, without selecting a sequence that is a part of the truncated Tat-responsive HIV promoter.

In some embodiments, the guide RNA can be a sequence complementary to a coding sequence such as a sequence encoding one or more viral structural proteins (e.g., gag, pol, env, and tat). Thus, the sequence can be complementary to sequence within the gag polyprotein, e.g., MA (matrix protein, p17); CA (capsid protein, p24); NC (nucleocapsid protein, p7); and P6 protein; pol, e.g., reverse transcriptase (RT) and RNase H, integrase (IN), and HIV protease (PR); env, e.g., gp160, or a cleavage product of gp160, e.g., gp120 or SU, and gp41 or TM; or tat, e.g., the 72-amino acid one-exon Tat or the 86-101 amino-acid two-exon Tat. In some embodiments, the guide RNA can be a sequence complementary to a sequence encoding an accessory protein, including for example, vif, n willef (negative factor) vpu (Virus protein U) and tev.

In some embodiments, the guide RNA sequence can be a sequence complementary to a structural or regulatory element (i.e., a target sequence) such as RRE, PE, SLIP, CRS (Cis-acting repressive sequences), and/or INS. RRE (Rev responsive element) is an RNA element encoded within the env region of HIV and includes approximately 200 nucleotides (positions 7710 to 8061 from the start of transcription in HIV-1, spanning the border of gp120 and gp41). PE (Psi element) corresponds to a set of 4 stem-loop structures preceding and overlapping the Gag start codon. SLIP is a TTTTTT “slippery site” followed by a stem-loop structure. CRS (Cis-acting repressive sequences). INS (Inhibitory/Instability RNA sequences) may be found for example, at nucleotides 414 to 631 in the gag region of HIV-1.

The guide RNA sequence can be a sense or anti-sense sequence. The guide RNA sequence generally includes a PAM. The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3) and Neiseria menigiditis requires 5′-NNNNGATT). The specific sequence of the guide RNA may vary, but, regardless of the sequence, useful guide RNA sequences will be those that minimize off-target effects while achieving high efficiency and complete ablation of the genomically integrated HIV provirus. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome including endogenous retroviral DNA, include bioinformatic screening using 12-bp+NGG target-selection criteria to exclude off-target human transcriptome or (even rarely) untranslated-genomic sites; avoiding transcription factor binding sites within the HIV-1 LTR promoter (potentially conserved in the host genome); selection of LTR-A- and -B-directed, 30-bp guide RNAs and also pre-crRNA system reflecting the original bacterial immune mechanism to enhance specificity/efficiency versus 20-bp guide RNA-, chimeric crRNA-tracRNA-based system and WGS, Sanger sequencing and SURVEYOR assay, to identify and exclude potential off-target effects.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs, for example a combination of sequences in U3, R, or U5, without selecting a sequence that is a part of the truncated Tat-responsive HIV promoter. When the compositions are administered in an expression vector, the guide RNAs can be encoded by a single vector. Alternatively, multiple vectors can be engineered to each include two or more different guide RNAs. Useful configurations will result in the excision of viral sequences between cleavage sites resulting in the ablation of HIV genome or HIV protein expression. Thus, the use of two or more different guide RNAs promotes excision of the viral sequences between the cleavage sites recognized by the CRISPR endonuclease. The excised region can vary in size from a single nucleotide to several thousand nucleotides. Exemplary excised regions are described in the examples.

When the compositions are administered as a nucleic acid or are contained within an expression vector, the CRISPR endonuclease can be encoded by the same nucleic acid or vector as the guide RNA sequences. Alternatively or in addition, the CRISPR endonuclease can be encoded in a physically separate nucleic acid from the guide RNA sequences or in a separate vector. In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-0-dimethylcytidine; 5-hydroxymethylcytidine; 5-formylcytidine; 2′-0-methyl-5-formaylcytidine; 3-methylcytidine; 2-thiocytidine; lysidine; 2′-0-methyluridine; 2-thiouridine; 2-thio-2′-O-methyluridine; 3,2′-0-dimethyluridine; 3-(3-amino-3-carboxypropyl)uridine; 4-thiouridine; ribosylthymine; 5,2′-0-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-0-methyluridine; 5-methoxycarbonylmethyl-T-thiouridine; 5-carbamoylmethyluridine; 5-carbamoylmethyl-2′-0-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-0-methyl-uridine; 5-carboxymethylaminomethyl-2-thiouridine; dihydrouridine; dihydroribosylthymine; 2′-methyladenosine; 2-methyladenosine; N.sup.6Nmethyladenosine; N⁶, N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-0-dimethyl inosine; 2′-O-methyl guanosine; 1-methyl guanosine; N²-methyl guanosine; N², N²-dimethyl guanosine; N², 2′-O-dimethyl guanosine; N², N², 2′-0-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

Isolated nucleic acid molecules can be produced by standard techniques. For example, PCR techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring portion of a Cas9-encoding DNA (in accordance with, for example, the formula above).

Two nucleic acids or the polypeptides they encode may be described as having a certain degree of identity to one another. For example, a Cas9 protein and a biologically active variant thereof may be described as exhibiting a certain degree of identity. Alignments may be assembled by locating short Cas9 sequences in the Protein Information Research (PIR) site (http://pir.georgetown.edu), followed by analysis with the “short nearly identical sequences” Basic Local Alignment Search Tool (BLAST) algorithm on the NCBI website (http://www.ncbi.nlm.nih.gov/blast).

A percent sequence identity to Cas9 can be determined and the identified variants may be utilized as a CRISPR-associated endonuclease and/or assayed for their efficacy as a pharmaceutical composition. A naturally occurring Cas9 can be the query sequence and a fragment of a Cas9 protein can be the subject sequence. Similarly, a fragment of a Cas9 protein can be the query sequence and a biologically active variant thereof can be the subject sequence. To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment). See Chenna et al., Nucleic Acids Res. 31:3497-3500, 2003.

Recombinant constructs are also provided herein and can be used to transform cells in order to express Cas9 under the control of a truncated Tat-responsive HIV LTR promoter. Recombinant constructs may similarly be utilized to express a guide RNA complementary to a target sequence in HIV. A recombinant nucleic acid construct comprises a nucleic acid encoding a Cas9 and/or a guide RNA complementary to a target sequence in HIV as described herein, operably linked to a regulatory region suitable for expressing the Cas9 and/or a guide RNA complementary to a target sequence in HIV in the cell. It will be appreciated that a number of nucleic acids can encode a polypeptide having a particular amino acid sequence. The degeneracy of the genetic code is well known in the art. For many amino acids, there is more than one nucleotide triplet that serves as the codon for the amino acid. For example, codons in the coding sequence for Cas9 can be modified such that optimal expression in a particular organism is obtained, using appropriate codon bias tables for that organism.

Nucleic acids as described herein may be contained in vectors. Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Several delivery methods may be utilized in conjunction with the truncated Tat-responsive HIV LTR promoter operably linked to the Cas9 gene for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005). An example non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors is known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Additional vectors include viral vectors, fusion proteins and chemical conjugates. Retroviral vectors include Moloney murine leukemia viruses and HIV-based viruses. One HIV based viral vector comprises at least two vectors wherein the gag and poi genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.: 90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See for example, Chen et al, BioTechniques, 34: 167-171 (2003), which is incorporated herein, by reference, in its entirety.

As described above, the compositions of the present invention can be prepared in a variety of ways known to one of ordinary skill in the art. Regardless of their original source or the manner in which they are obtained, the compositions disclosed herein can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having an HIV infection or at risk for contracting and HIV infection. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

The pharmaceutical compositions may contain, as the active ingredient, nucleic acids and vectors described herein in combination with one or more pharmaceutically acceptable carriers. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or coincorporated with tissue-specific antibodies, for example antibodies that target cell types that are commonly latently infected reservoirs of HIV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding a CRISPR-associated endonuclease and optionally a guide RNA is operably linked to the truncated Tat-responsive HIV LTR promoter as described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycolmodiifed (PEGylated) low molecular weight LPEI.

The nucleic acids and vectors may also be applied to a surface of a device (e.g., a catheter) or contained within a pump, patch, or other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions may be formulated as a topical gel for blocking sexual transmission of HIV. The topical gel can be applied directly to the skin or mucous membranes of the male or female genital region prior to sexual activity. Alternatively or in addition the topical gel can be applied to the surface or contained within a male or female condom or diaphragm.

In some embodiments, the compositions can be formulated as a nanoparticle encapsulating a nucleic acid encoding Cas9 or a variant Cas9 operably linked to a truncated HIV LTR promoter. The nucleic acid may additionally encode a guide RNA sequence complementary to a target HIV.

The present formulations can encompass a vector encoding Cas9 and a guide RNA sequence complementary to a target HIV. The guide RNA sequence can include a sequence complementary to a single target region or it can include any combination of sequences complementary to multiple target regions as described earlier. Alternatively the sequence encoding Cas9 driven by the truncated HIV LTR promoter and the sequence encoding the guide RNA sequence can be on separate vectors.

The compositions disclosed herein are generally and variously useful for treatment of a subject having an HIV infection. The methods are useful for targeting any HIV, for example, HIV-1 and HIV-2, and also SIV, and any circulating recombinant form thereof. A subject is effectively treated whenever a clinically beneficial result ensues. This may mean, for example, a complete resolution of the symptoms of a disease, a decrease in the severity of the symptoms of the disease, or a slowing of the disease's progression. These methods can further include the steps of a) identifying a subject (e.g., a patient and, more specifically, a human patient) who has an HIV infection; and b) providing to the subject a composition comprising a nucleic acid encoding a CRISPR-associated nuclease, e.g., Cas9, under control of the truncated Tat-responsive HIV LTR promoter. The methods may further include providing to the subject a sequence encoding a guide RNA complementary to an HIV target sequence, e.g. an HIV LTR.

A subject can be identified using standard clinical tests, for example, immunoassays to detect the presence of HIV antibodies or the HIV polypeptide p24 in the subject's serum, or through HIV nucleic acid amplification assays. An amount of such a composition provided to the subject that results in a complete resolution of the symptoms of the infection, a decrease in the severity of the symptoms of the infection, or a slowing of the infection's progression is considered a therapeutically effective amount. The present methods may also include a monitoring step to help optimize dosing and scheduling as well as predict outcome. In some methods of the present invention, one can first determine whether a patient has a latent HIV infection, and then make a determination as to whether or not to treat the patient with one or more of the compositions described herein. Monitoring can also be used to detect the onset of drug resistance and to rapidly distinguish responsive patients from nonresponsive patients. In some embodiments, the methods can further include the step of determining the nucleic acid sequence of the particular HIV harbored by the patient and then designing the guide RNA to be complementary to those particular sequences. For example, one can determine the nucleic acid sequence of a subject's LTR U3, R, or U5 region and then design one or more guide RNAs to be precisely complementary to the patient's sequences, again without selecting a sequence that is a part of the truncated Tat-responsive HIV promoter.

The compositions are also useful for the treatment, for example, as a prophylactic treatment, of a subject at risk for having a retroviral infection, e.g., an HIV infection. These methods can further include the steps of a) identifying a subject at risk for having an HIV infection; b) providing to the subject a composition comprising a nucleic acid encoding a CRISPR-associated nuclease, e.g., Cas9, under control of a truncated Tat-responsive HIV-1 LTR promoter. The sequence may additionally encode for a guide RNA complementary to an HIV target sequence, e.g. an HIV LTR. A subject at risk for having an HIV infection can be, for example, any sexually active individual engaging in unprotected sex, i.e., engaging in sexual activity without the use of a condom; a sexually active individual having another sexually transmitted infection; an intravenous drug user; or an uncircumcised man. A subject at risk for having an HIV infection can be, for example, an individual whose occupation may bring him or her into contact with HIV-infected populations, e.g., healthcare workers or first responders. A subject at risk for having an HIV infection can be, for example, an inmate in a correctional setting or a sex worker, that is, an individual who uses sexual activity for income employment or nonmonetary items such as food, drugs, or shelter.

The compositions can also be administered to a pregnant or lactating woman having an HIV infection in order to reduce the likelihood of transmission of HIV from the mother to her offspring. A pregnant woman infected with HIV can pass the virus to her offspring transplacentally in utero, at the time of delivery through the birth canal or following delivery, through breast milk. The compositions disclosed herein can be administered to the HIV infected mother either prenatally, perinatally or postnatally during the breast-feeding period, or any combination of prenatal, perinatal, and postnatal administration. Compositions can be administered to the mother along with standard antiretroviral therapies as described below. In some embodiments, the compositions of the invention are also administered to the infant immediately following delivery and, in some embodiments, at intervals thereafter. The infant also can receive standard antiretroviral therapy.

The compositions may be administered to an individual who is not infected with HIV to prevent infection with HIV. The composition may include delivering a therapeutically effective amount of the pharmaceutical composition. The pharmaceutical composition may include a sequence encoding a CRISPR-associated endonuclease and at least the core region of a HIV LTR promoter and a TAR region of the truncated Tat-responsive HIV LTR promoter as described above.

The methods disclosed herein can be applied to a wide range of species, e.g., humans, non-human primates (e.g., monkeys), horses or other livestock, dogs, cats, ferrets or other mammals kept as pets, rats, mice, or other laboratory animals.

The methods of the invention can be expressed in terms of the preparation of a medicament. Accordingly, the invention encompasses the use of the agents and compositions described herein in the preparation of a medicament. The compounds described herein are useful in therapeutic compositions and regimens or for the manufacture of a medicament for use in treatment of diseases or conditions as described herein.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. An effective amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Any method known to those in the art can be used to determine if a particular response is induced. Clinical methods that can assess the degree of a particular disease state can be used to determine if a response is induced. The particular methods used to evaluate a response will depend upon the nature of the patient's disorder, the patient's age, and sex, other drugs being administered, and the judgment of the attending clinician.

The compositions may also be administered with another therapeutic agent, for example, an anti-retroviral agent, used in HAART. Antiretroviral agents may include reverse transcriptase inhibitors (e.g., nucleoside/nucleotide reverse transcriptase inhibitors, zidovudine, emtricitibine, lamivudine and tenoifvir; and non-nucleoside reverse transcriptase inhibitors such as efavarenz, nevirapine, rilpivirine); protease inhibitors, e.g., tipiravir, darunavir, indinavir; entry inhibitors, e.g., maraviroc; fusion inhibitors, e.g., enfuviritide; or integrase inhibitors e.g., raltegrivir, dolutegravir. Antiretroviral agents may also include multi-class combination agents for example, combinations of emtricitabine, efavarenz, and tenofivir; combinations of emtricitabine; rilpivirine, and tenofivir; or combinations of elvitegravir, cobicistat, emtricitabine and tenofivir.

Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks. The therapeutic agents may be administered under a metronomic regimen, e.g., continuous low-doses of a therapeutic agent.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue specific antibody. The liposomes will be targeted to and taken up selectively by the organ.

Also provided, are methods of inactivating a retrovirus, for example a lentivirus such as a human immunodeficiency virus, a simian immunodeficiency virus, a feline immunodeficiency virus, or a bovine immunodeficiency virus in a mammalian cell. The human immunodeficiency virus can be HIV-1 or HIV-2. The human immunodeficiency virus can be a chromosomally integrated provirus. The mammalian cell can be any cell type infected by HIV, including, but not limited to CD4⁺ lymphocytes, macrophages, fibroblasts, monocytes, T lymphocytes, B lymphocytes, natural killer cells, dendritic cells such as Langerhans cells and follicular dendritic cells, hematopoietic stem cells, endothelial cells, brain microglial cells, astrocytes and gastrointestinal epithelial cells. Such cell types include those cell types that are typically infected during a primary infection, for example, a CD4⁺ lymphocyte, a macrophage, a monocyte or a Langerhans cell, as well as those cell types that make up latent HIV reservoirs, i.e., a latently infected cell.

The methods can include exposing and/or contacting the cell to a composition comprising an isolated nucleic acid encoding a CRISPR-associated endonuclease operably linked to a truncated HIV LTR promoter containing the core region and the TAR region of the HIV LTR promoter. The isolated nucleic acid may further encode one or more guide RNAs wherein the guide RNA is complementary to a target nucleic acid sequence in the retrovirus. The contacting step can take place in vivo, that is, the compositions can be administered directly to a subject having HIV infection. The methods are not so limited however, and the contacting step can take place ex vivo. For example, a cell or plurality of cells, or a tissue explant, can be removed from a subject having an HIV infection and placed in culture, and then contacted with a composition comprising a CRISPR-associated endonuclease operably linked to a truncated HIV LTR promoter and optionally a guide RNA wherein the guide RNA is complementary to a nucleic acid sequence in HIV. As described above, a pharmaceutical composition may include a nucleic acid encoding a CRISPR-associated endonuclease operably linked to a truncated Tat-responsive HIV LTR promoter.

The compositions are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

Standard methods, for example, immunoassays to detect the CRISPR-associated endonuclease, or nucleic acid-based assays such as PCR to detect the guide RNA, can be used to confirm cell has taken up and/or expressed the protein into which it has been introduced. The engineered cells can then be reintroduced into the subject from whom they were derived as described below.

In other embodiments, the compositions comprise a cell which has been transformed or transfected with one or more Cas9/truncated Tat-responsive HIV LTR promoter vectors. In some embodiments, the methods of the invention can be applied ex vivo. That is, a subject's cells can be removed from the body and treated with the compositions in culture to excise HIV sequences and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

The compositions described herein can be packaged in suitable containers labeled, for example, for use as a therapy to treat a subject having a retroviral infection, for example, an HIV infection or a subject at for contracting a retroviral infection, for example, an HIV infection. The containers can include a composition comprising a nucleic acid sequence encoding a CRISPR-associated endonuclease, for example, a Cas9 endonuclease, and a truncated Tat-responsive HIV LTR promoter as described earlier. The sequence may additionally encode a guide RNA complementary to a target sequence in a HIV, or a vector encoding that nucleic acid, and one or more of a suitable stabilizer, carrier molecule, flavoring, and/or the like, as appropriate for the intended use. Accordingly, packaged products (e.g., sterile containers containing one or more of the compositions described herein and packaged for storage, shipment, or sale at concentrated or ready-to-use concentrations) and kits, including at least one of the disclosed compositions. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required. In some embodiments, the kits can include one or more additional antiretroviral agents, for example, a reverse transcriptase inhibitor, a protease inhibitor or an entry inhibitor. The additional agents can be packaged together in the same container as a nucleic acid sequence encoding a CRISPR-associated endonuclease, for example, a Cas9 endonuclease, operably linked to a truncated HIV LTR promoter and optionally a guide RNA complementary to a target sequence in a HIV, or a vector encoding that nucleic acid or they can be packaged separately.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

The practice of the invention is illustrated by the following non-limiting examples.

### EXAMPLES

### Example 1: Cloning of LTR-Cas9 Variants

Full length and various truncated LTR promoter sequences were obtained by PCR using pNL4-3 HIV vector (NIH AIDS Reagent Program #114) as a template and the following primers (restriction sites noted in boldface):

(SEQ ID NO: 1)

| | |
|---|---|
| Kpn1-LTR(−454)-S | 5′-GGTACCTGGAAGGGCTAATTTGG-3′ |

(SEQ ID NO: 2)

| | |
|---|---|
| Kpn1-LTR(−120)-S | 5′-GGTACCTCGAGCTTTCTACAAGG-3′ |

(SEQ ID NO: 3)

| | |
|---|---|
| Xba1-LTR(−80)-S | 5′-TCTAGAGGAGGTGTGGCCTGGGC-3′ |

(SEQ ID NO: 4)

| | |
|---|---|
| Kpn1-LTR(−38)-S | 5′-GGTACCAGATGCTACATATAAGC-3, |
| or | |

(SEQ ID NO: 5)

| | |
|---|---|
| LTR(+66)-Nco1-AS | 5′-CCATGGTAAGCAGTGGGTTCC-3′. |

The derivation of the truncated HIV-1 LTR promoter variants is shown in diagrammatically in FIG. 1A with reference to the U3, R and U5 regions of the LTR, and the LTR enhancer, core, TAR (trans-activation-responsive) and TATA box elements. FIG. 1B shows an agarose gel electrophoresis image of the PCR-amplified LTR truncation variants.

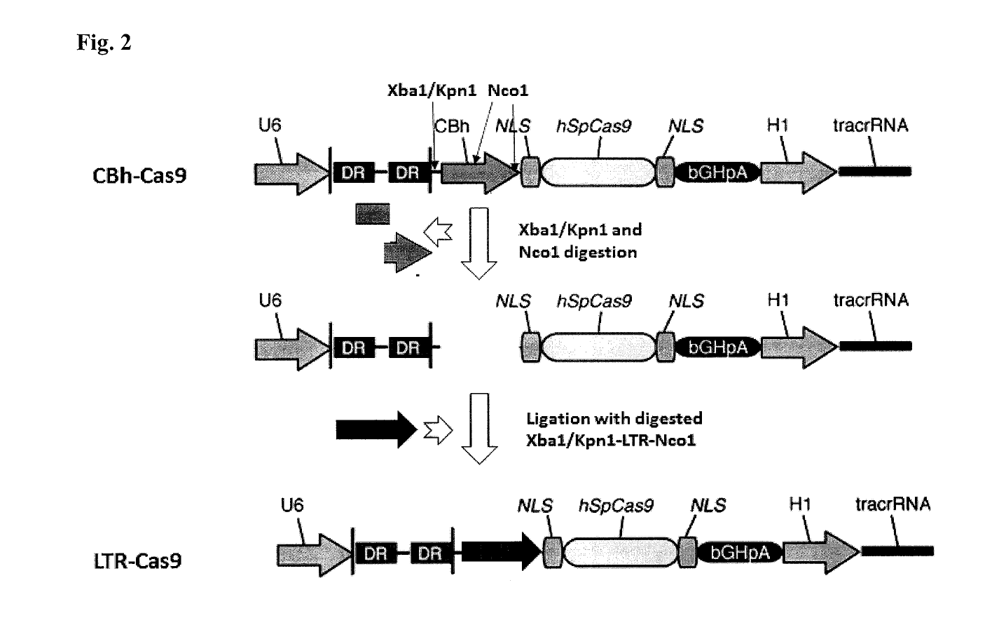

PCR products were gel purified and directly subcloned in TA vector (Invitrogen), then excised with Kpnl or Xbal and Ncol restriction enzymes and ligated into Kpnl-Ncol or Xbal-Ncol digested pX260-U6-DR-BB-DR-Cbh-NLS-hSpCas9-NLS-H1-shorttracr-PGK-puro plasmid (Addgene #42229) (hereinafter “pX260 plasmid”) as a Cas9 gene source/template. The pX260 plasmid contains a Cbh promoter (Xbal-Kpnl-Cbh-Ncol) As a result of the manipulation, the original Cbh promoter in the pX260 plasmid was removed and replaced with one of the LTR promoters (Xbal- or Kpnl-LTR-Ncol). A blueprint of the original pX260 plasmid structure in shown in FIG. 2, identified as “Cbh-Cas9” (from www.Addagene.org and Cong et al., Science (2013) 339(6121):819-23). A blueprint of a modified plasmid is shown in FIG. 2 as “LTR-Cas9”.

### Example 2: Optimization of LTR/Tat Ratio for Inducing Cas9 Expression

To find an optimal ratio between Tat and LTR promoter for the best transactivation effect, cells of the human primary glioblastoma cell line U87 MG were co-transfected using Lipofectamine 2000 reagent (Invitrogen) with different amounts of plasmid expressing FLAG-labeled Cas9 under control of full length HIV-1 LTR (pLTR(−454/+66)-FLAG-Cas9) plasmid (10, 50 and 250 ng), with or without Tat expressing plasmid (pCMV-Tat86, 250 ng). U87 MG is an HIV-1 latency reporter cell line. The total amount of DNA was equilibrated with empty pCMV plasmid (pcDNA3.1). Forty-eight hours later, cells were lysed in TNN buffer (50 mM Tris pH 7.4, 100 mM NaCl, 5 mM EDTA, 1% NP 40). Cas9, Tat and α-tubulin expression were then examined by Western blot. The results are shown in FIG. 3A (U87 MG WCE 50 μg/well). Lane 1: pLTR(−454/+66)-Cas9 250 ng, pCMV 1000 ng. Lane 2: pLTR(−454/+66)-Cas9 50 ng, pCMV 1200 ng. Lane 3: pLTR(−454/+66)-Cas9 10 ng, pCMV 1240 ng. Lane 4: pLTR(−454/+66)-Cas9 250 ng, pCMV 750 ng, pCMV-Tat86 250 ng. Lane 5: pLTR(−454/+66)-Cas9 50 ng, pCMV 950 ng, pCMV-Tat86 250 ng. Lane 6: pLTR(−454/+66)-Cas9 10 ng, pCMV 990 ng, pCMV-Tat86 250 ng.

The intensity of bands corresponding to Cas9 and α-tubulin (used as a loading control) were analyzed and compared using ImageJ software. The results are shown in FIG. 3B. The top panel shows the Western blot image quantification of the Cas9 levels normalized to the levels of α-tubulin, with or without Tat. The bottom panel show the Western blot image quantification of the +Tat/no Tat ratio. The results indicate that maximal (5.3×) induction of Cas9 expression was obtained at a 1:5 ratio of pLTR-Cas9:pCMVTat86 (50 ng:250 ng).

### Example 3: Comparison of Truncated LTR Promoters in Inducing Cas9 Expression

To test and compare truncated LTR promoters, U87 MG cells were transfected with different amounts of plasmids (5 ng or 50 ng) expressing FLAG-labeled Cas9 under control of the HIV-1 truncated LTR variant pLTR(−120/+66)-FLAG-Cas9 or the HIV-1 truncated LTR variant pLTR(−80/+66)-FLAG-Cas9, with or without Tat expressing plasmid (pCMV-Tat86, 250 ng). Forty-eight hours later, whole cell lysates where prepared and resolved by Western blot. Intensity of bands corresponding to Cas9 and α-tubulin (used as a loading control) were analyzed and compared using ImageJ software. The results are shown in FIG. 4A. Lane 1: pLTR(−120/+66)-Cas9 5 ng, pCMV 1245 ng. Lane 2: pLTR(−120/+66)-Cas9 5 ng, pCMV 1245 ng, +rTat protein 2.5 μg/ml. Lane 3: pLTR(−120/+66)-Cas9 5 ng, pCMV 995 ng, pCMV-Tat86 250 ng. Lane 4: pLTR(−120/+66)-Cas9 50 ng, pCMV 1200 ng. Lane 5: pLTR(−120/+66)-Cas9 50 ng, pCMV 1200 ng, +rTat protein 2.5 μg/ml. Lane 6: pLTR(−120/+66)-Cas9 50 ng, pCMV 950 ng, pCMV-Tat86 250 ng. Lane 7: pLTR(−80/+66)-Cas9 5 ng, pCMV 1245 ng. Lane 8: pLTR(−80/+66)-Cas9 5 ng, pCMV 1245 ng, +rTat protein 2.5 μg/ml. Lane 9: pLTR(−80/+66)-Cas9 5 ng, pCMV 995 ng, pCMV-Tat86 250 ng. Lane 10: pLTR(−80/+66)-Cas9 50 ng, pCMV 1200 ng. Lane 11: pLTR(−80/+66)-Cas9 50 ng, pCMV 1200 ng, +rTat protein 2.5 μg/ml. Lane 12: pLTR(−80/+66)-Cas9 50 ng, pCMV 950 ng, pCMV-Tat86 250 ng.

The intensity of bands corresponding to Cas9 and α-tubulin (used as a loading control) were analyzed and compared using ImageJ software. The results are shown in FIG. 4B. The top panel show the Western blot image quantification of the Cas9 levels normalized to the levels of α-tubulin, with no Tat, with rTAT or with transfected Tat. The bottom panel show the Western blot image quantification of the +Tat(transfected)/no Tat ratio. The results demonstrate that removing modulatory and/or enhancer regions of the LTR (those regions being schematically represented in FIG. 1A) did not significantly affect Tat-mediated transactivation of Cas9 expression. Tat-mediated expression was apparent from the pLTR(−80/+66)-FLAG-Cas9 plasmid, containing the core and TAR LTR promoters elements, but not the enhancer and modulatory regions.

### Example 4: Negative Feedback Regulation of HIV-1 by Gene Editing Strategy

In the studies presented here, the gene editing composition allows conditional activation of the CRISPR/Cas9 at the early stage of viral reactivation by the HIV-1 transcriptional activator, Tat. This new strategy completely and permanently ablates virus replication prior to productive viral replication by removing a segment of the viral gene spanning the viral promoter and/or the viral coding sequence. Further, this strategy alleviates any concerns due to unforeseen complications that may arise by unnecessary and persistent expression of Cas9 at high levels in cells.

Results

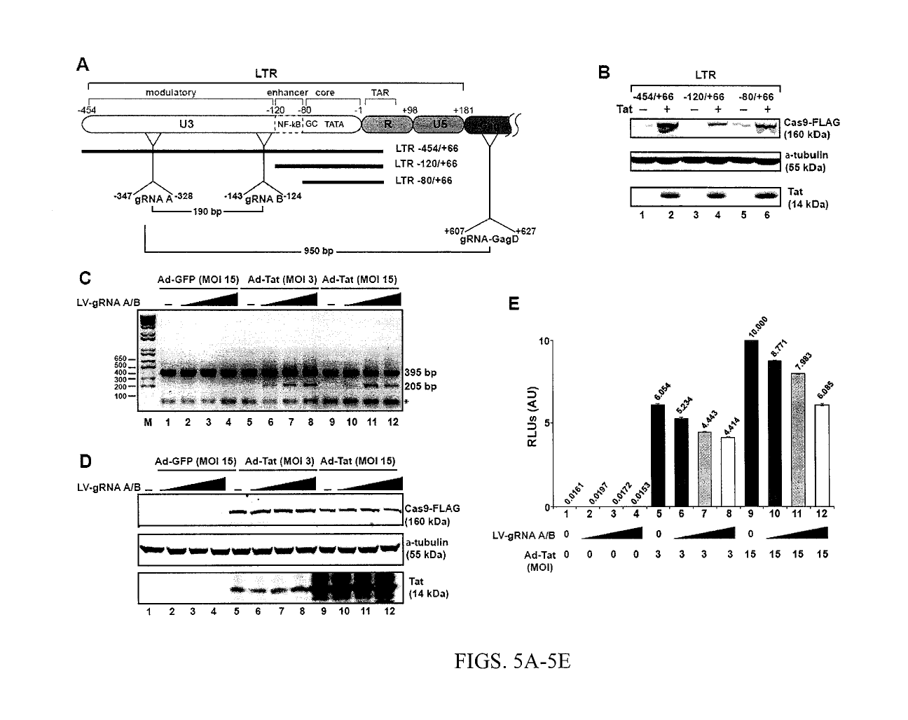

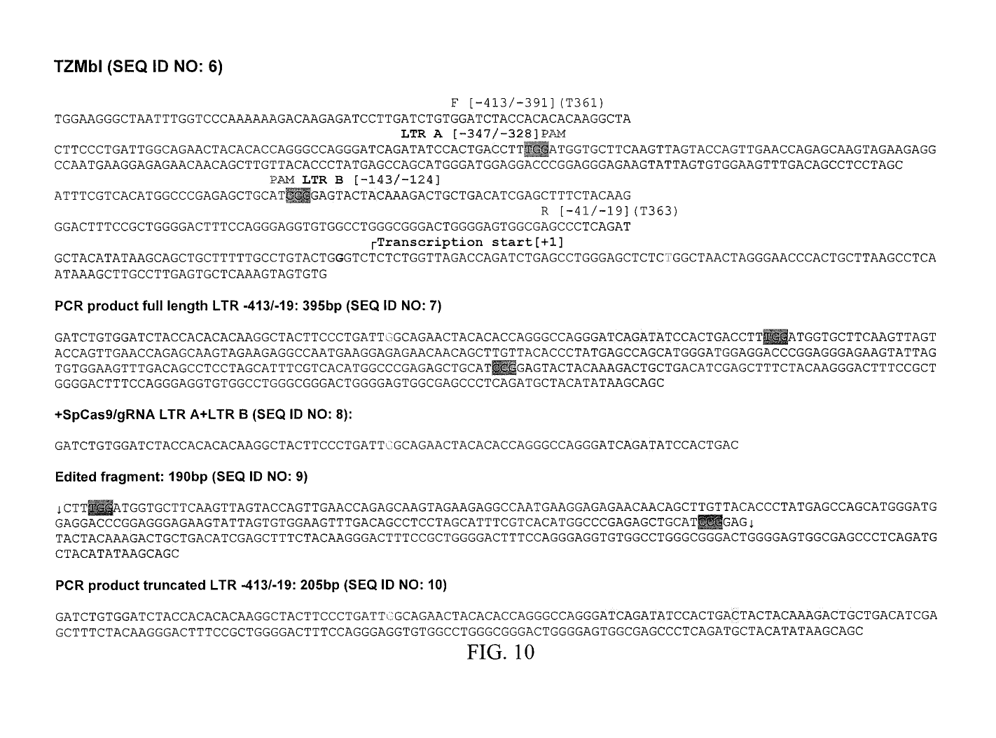

The coding DNA sequence corresponding to the Cas9 gene was placed in a pX26 expression vector plasmid containing three different segments of the HIV-1 promoter spanning the U3 and R regions of the 5′-LTR to identify the minimal DNA elements of the viral promoter that remain responsive to Tat, yet lacks the sequences corresponding to gRNAs A and B that are initially used for editing HIV-1 DNA (FIG. 5A). After verification of this cloning strategy by DNA sequencing of each construct, expression of Cas9 by each vector and the level of responsiveness to Tat was examined in TZMb1 cells co-transfected with pX26 or pX26-LTR-Cas9 and CMV-Tat. Results from Western blot revealed activation of expression of Cas9 by all three constructs including the plasmid encompassing the minimal DNA promoter sequence positioned between −80 to +66 (FIG. 5B). This was particularly important for these studies as the promoter sequence resides outside of the DNA sequences corresponding to gRNAs A and B (FIG. 5B). Next, a DNA fragment corresponding to LTR_{(-80/+66)}-Cas9 was cloned into a lentiviral vector (LV) and used to transduce TZMb1 cells to assess the effect of Tat protein on the editing of integrated copies of HIV-1 DNA expressing the luciferase reporter gene. Results from PCR amplification of the LTR revealed the detection of 205 bp DNA fragment in cells expressing gRNAs A and B and Tat protein (FIG. 5C, compare lanes 1-5 to lanes 6-8). The position of the primers used for PCR amplification and the expected amplicons are illustrated in FIG. 5A (also see FIG. 10). Results from sequencing verified excision of the 190 bp DNA fragment upon expression of Tat in cells transduced by LV-LTR_{(-80/+66)}-Cas9 plus LV-gRNAs A/B. Expression of Cas9, Tat and α-tubulin (control for equal loading) are shown in FIG. 5D.

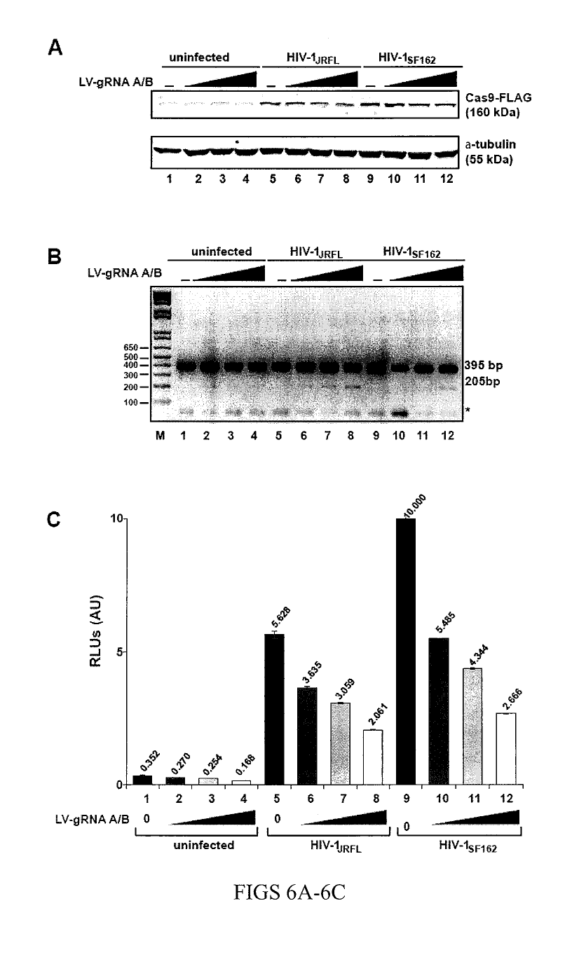

Next, the impact of the viral DNA excision on viral promoter activity was examined by luciferase assay. Results show a gradual decrease in luciferase activity upon activation of Cas9 by Tat, corroborating the results from DNA assay, indicating that the cleavage of DNA causes inhibition of viral promoter activity in these cells (FIG. 5E). In follow-up studies, the activation of Cas9 was investigated upon infection of TZMb1 cells by HIV-1. To this end, cells were transduced by LV-LTR_{(-80/+66)}-Cas9 and LV-gRNAs A/B for 24 hours, after which cells were infected with HIV-1_{JRFL} or HIV-1_{SF162} at three different MOIs. After 48 hours, cells were harvested for evaluating DNA excision by PCR, expression of the integrated promoter sequence by luciferase assay, and expression of Cas9 by Western blot. Results from these experiments show the detection of a post-cleavage 205 bp DNA fragment in cells infected with HIV-1_{JRFL} and HIV-1_{SF162}, indicating that production of Tat by HIV-1_{JRFL} and HIV-1_{SF162} transactivated the LTR_{(-80/+66)} promoter and production of Cas9 in these cells (FIG. 6A). Further, results from the luciferase assay revealed significant reduction of luciferase activity in the cells, again verifying the effectiveness of Cas9 activation by Tat, which is produced upon infection by HIV-1_{JRFL} or HIV-1_{SF162} in shutting down the integrated HIV-1 luciferase gene. Induction of Cas9 in the infected cells is shown in FIG. 6B. Results from Western blot showed activation of the truncated LTR promoter, LTR_{(-80/+66)}, upon infection of cells with HIV-1_{JRFL} and HIV-1_{SF162}, resulting in the production of Cas9 protein in the cells (FIG. 6C).

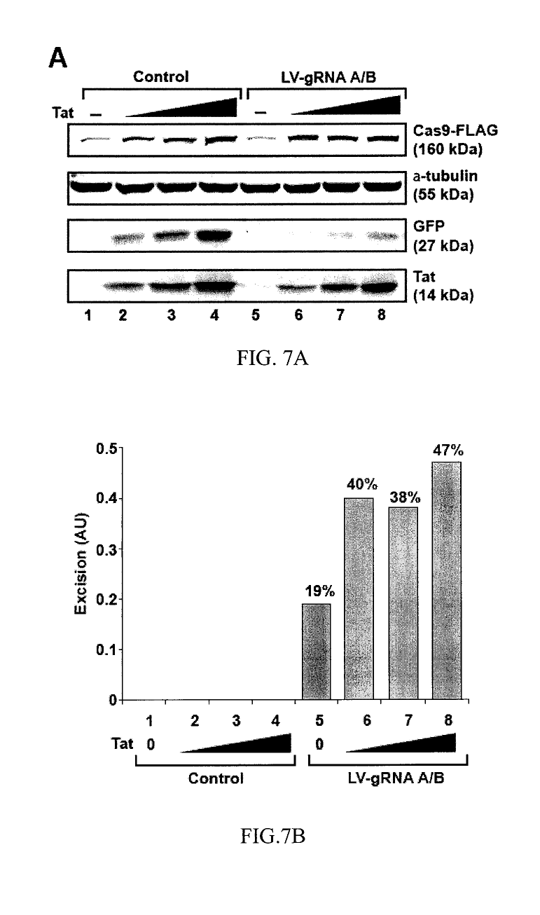

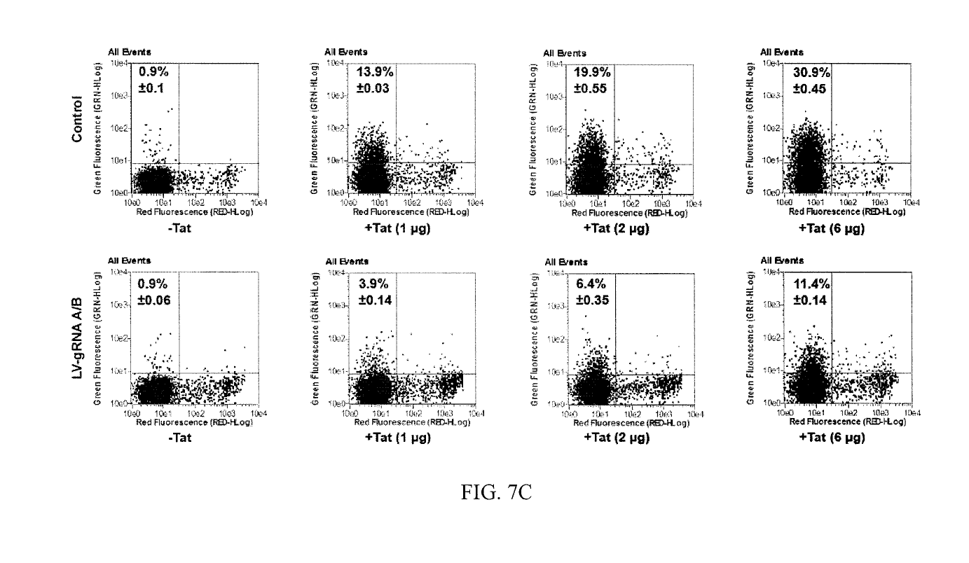

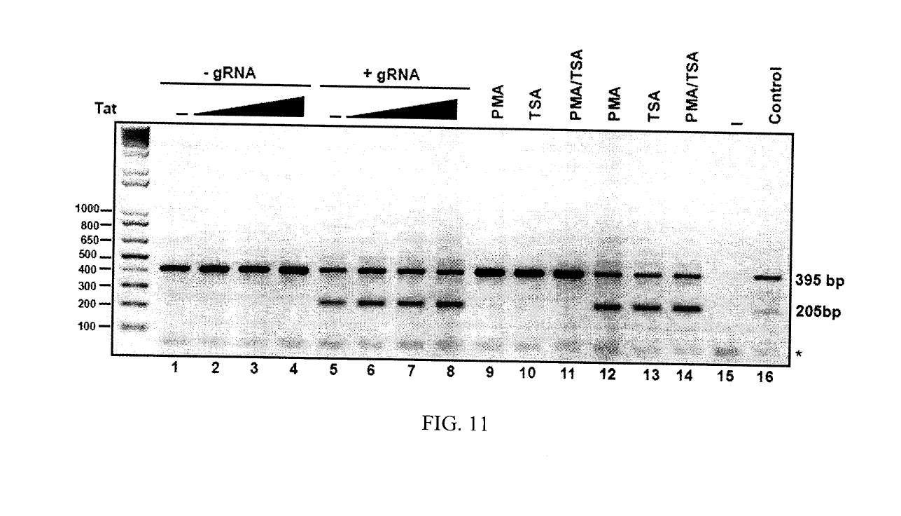

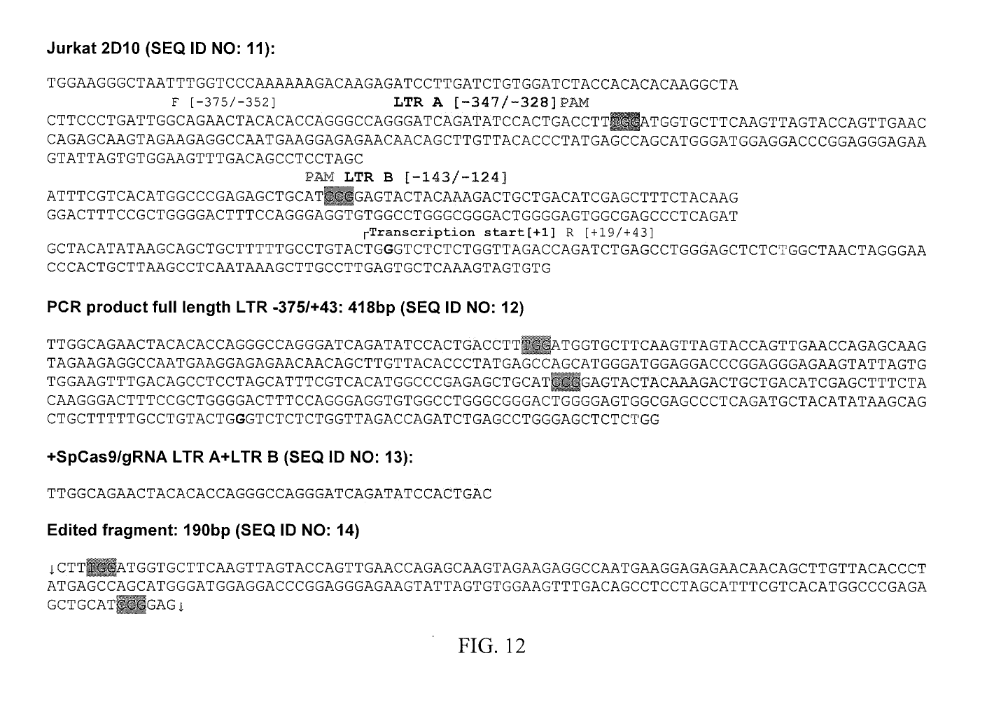

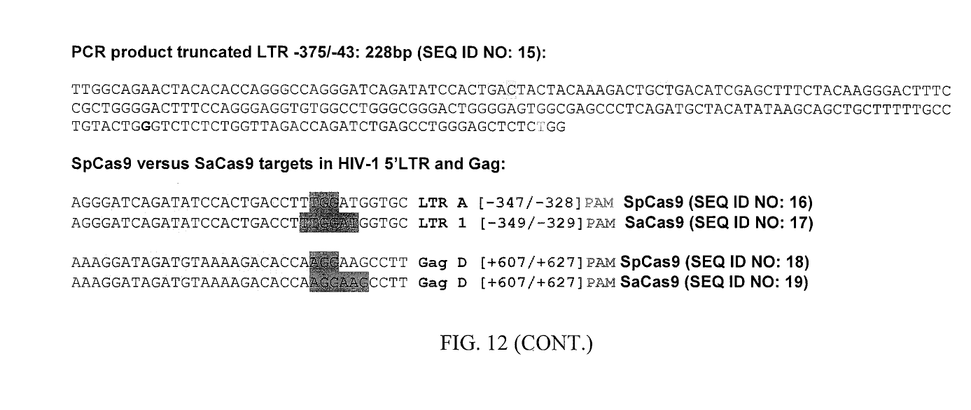

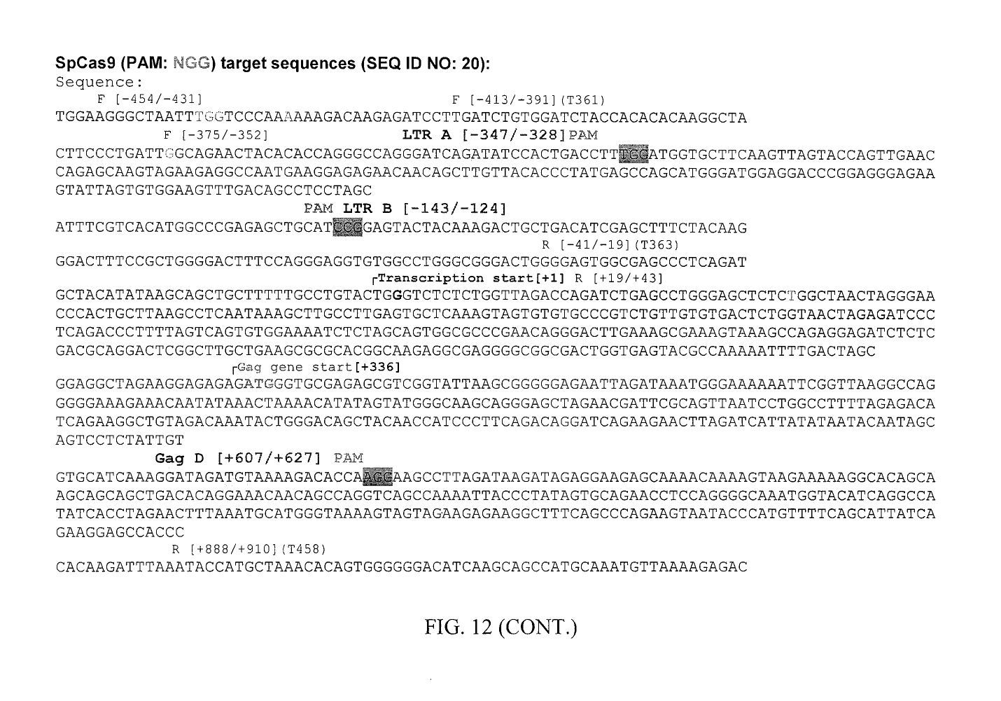

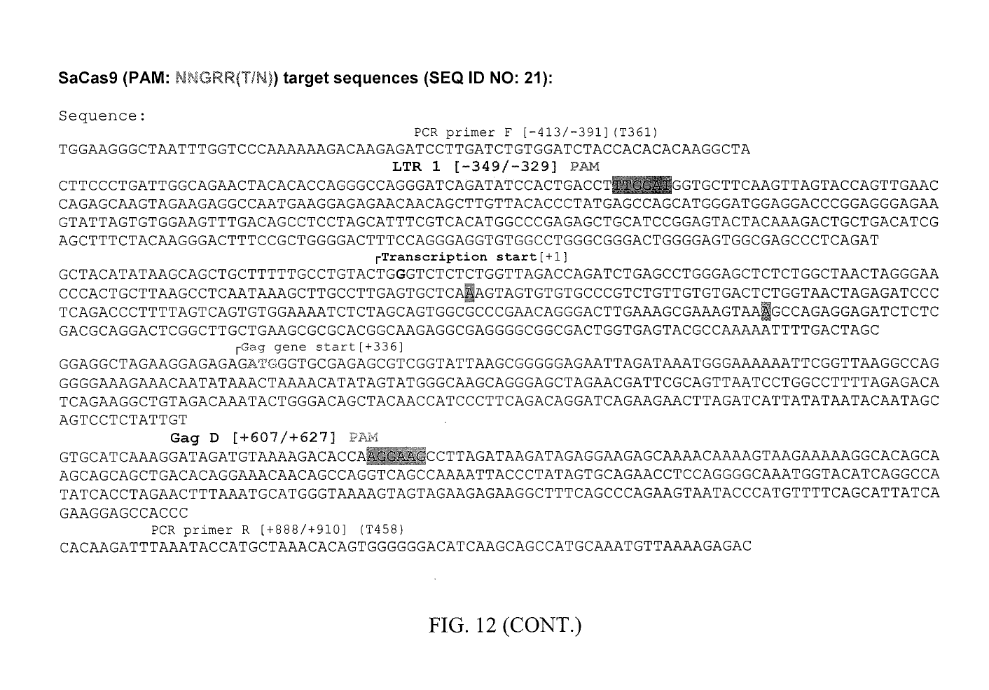

In the follow-up, the ability of Tat-mediated activation of the LTR-Cas9 was tested along with gRNAs A/B in eliminating the HIV-1 genome in the human T-lymphocytic cells line, 2D10. These cells harbor integrated copies of a single round HIV-1PNLA4-3 in a latent state, whose genome lacks a portion of the Gag and Pol genes and the Nef gene is replaced by a gene encoding the reporter green fluorescent protein (GFP). The enhanced level of Tat protein in these cells and the activation of Cas9 (shown in FIG. 7A) caused editing of the viral LTR upon activation of Cas9 in the cells transduced by LV-gRNAs A/B (FIG. 7B, also see FIG. 11, lanes 1-8). Accordingly, a significant decrease in the number of GFP positive cells was detected in the presence of Tat, indicating that activation of Tat eliminates the capacity of the cleaved promoter in expressing viral DNA, which in turn, causes suppression of GFP in these cells. The DNA sequence corresponding to the position of the gRNAs, excision of the DNA fragment and PCR primers are shown in FIG. 12.

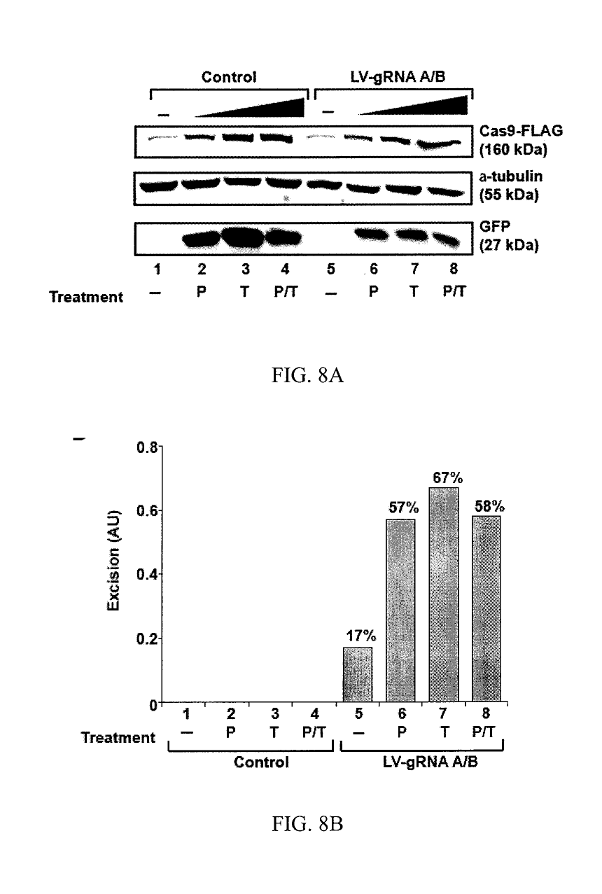

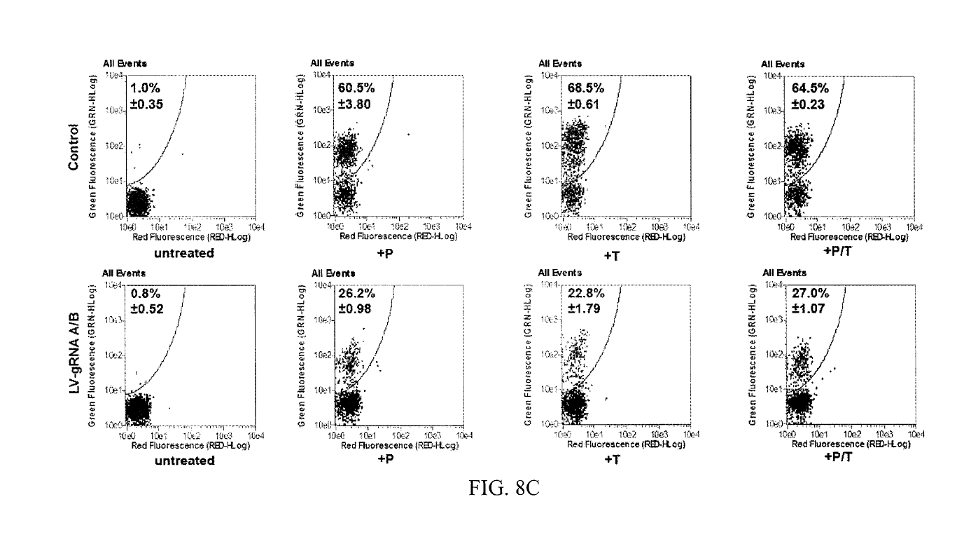

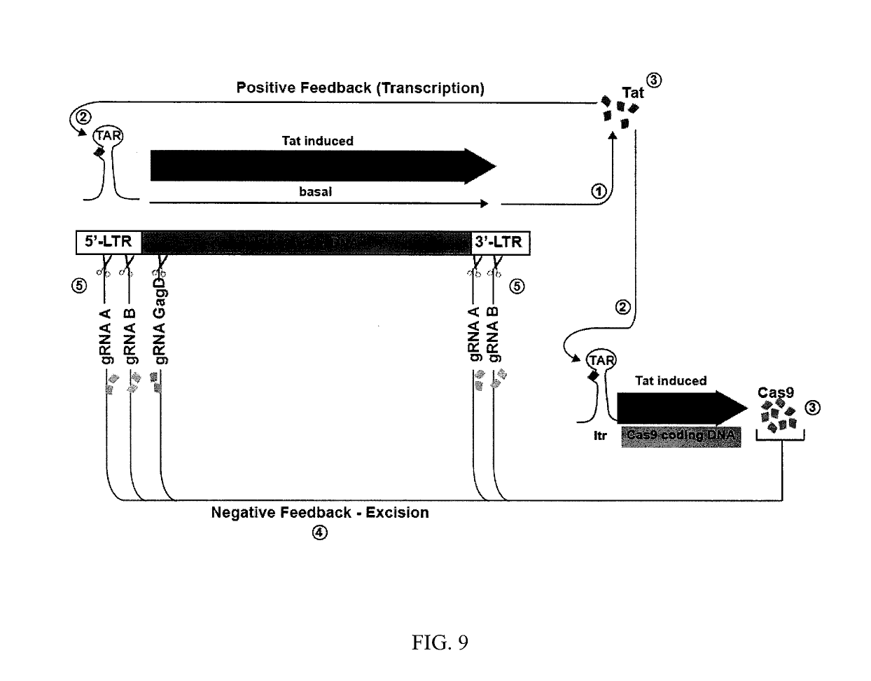

In light of earlier observations indicating to the ability of PMA and/or TSA in stimulating integrated copies of proviral DNA in 2D10 cells, the impact of PMA and TSA on the activation of Cas9 in a latently infected T-cell model was assessed. As seen in FIG. 8A, treatment of 2D10 cells with PMA and TSA, singly or in combination, increased the level of Cas9 expression. In a parallel experiment, PCR analysis was performed for the detection of LTR DNA and showed a clear increase in the level of viral DNA excision (FIG. 8B), as evidenced by the appearance of the 205 bp DNA fragment (see FIG. 11, lanes 9-14). Examination of viral activation by measuring the level of GFP in the cells using Western blot or the quantification of green fluorescent cells, indicative of viral activation, by fluorescent microscopy (FIG. 8C) showed a drastic decrease in the level of viral gene expression. Thus, it is likely that activation of the minimal viral promoter (−88/+60) either by Tat produced upon reactivation of the silent provirus or directly by PMA and TSA that produced Cas9, have a negative impact on the expression of the latently integrated viral genome in cells containing gRNAs A and B.

### DISCUSSION

Since its discovery in 1985, the Tat protein of HIV-1 has captured significant attention due to its critical role in expression of the viral genome at the transcriptional level and its pathogenic impact on uninfected cells. Mechanistically, Tat associates with the RNA sequence located downstream of the initiation site from transcription (nucleotides +1 to +59), so-called transcription responsive region or TAR. The association of Tat with TAR triggers a series of molecular and biochemical events leading to the formation of pre-initiation and initiation complexes of transcription in proximity to the transcription start site (nucleotide +1). This complex includes a series of cellular proteins that have the ability to phosphorylate or acetylate components of the complexes including pTEF and RNA polymerase II, thus facilitating transcriptional elongation of RNA. In addition, the interaction of Tat with various transcriptional factors including NF-κB, p300/CBP and GCN5 can affect transcription of other viral and cellular genes; all of which contribute to the disease spectrum seen in HIV-1 positive AIDS patients. Tat also plays a major role in the productive replication of latent virus in reservoirs once transcription from the reactivated viral promoter leads to an initial round of viral transcription and Tat production. The unique importance of Tat in HIV-1 replication and the pathogenesis of AIDS, provided a strong rationale for serving as a potential target for drug discovery as well as vaccine development. In fact, several potent inhibitors, some with the ability to interfere with Tat-TAR interaction and others with the capacity to prevent Tat communication with its cellular partners, have shown various degrees of efficacy in affecting HIV-1 replication.

The strategy that was utilized in this study was to recruit Tat to excise a segment of the viral genome and permanently ablate HIV-1 gene transcription and replication in cells with productive or latent HIV-1. Here a suicide path was designed for HIV-1 that is triggered by Tat and includes editing of the viral genome using CRISPR/Cas9 technology (illustrated in FIG. 9). According to this pathway, production of Tat in the cells, in addition to stimulating its own promoter with the full-length 5′-LTR sequence, potentiates expression of Cas9 through the same mechanism by a truncated minimal promoter sequence spanning the GC-rich, TATA box, and TAR (−80 to +66) regions. Production of Cas9 and its association with gRNAs designed to target the LTR DNA sequence outside of the (−80 to +66) induced InDel mutations within the full-length viral promoter and by excising a segment of the gene, can permanently eradicate HIV-1 in the cells. In addition to the expected 417 bp DNA fragment representing the full-length LTR sequence, results from short-range amplification of LTR DNA showed a second DNA fragment of 227 bp in size only in cells expressing Tat. The 227 bp DNA fragment was generated by joining the residual 5′-LTR to the remaining 3′-LTR after cleavage by Cas9/gRNA A at either the 5′-LTR or the 3′-LTR. It is also likely that ligation of the remaining DNA fragment from the 5′-LTR with those from the 3′-LTR after cleavage by Cas9/gRNA created a new template for gene amplification and the appearance of a similar size (227 bp) amplicons. A multiplex of gRNAs were utilized that target the LTR (gRNA A) plus a region within the Gag gene with the expectation of the removal of DNA fragment between gRNA A and gRNA Gag.

The CRISPR/Cas9 gene editing strategy has received attention in biomedical research in recent years due to its extraordinary ability to edit the genome with precision and high efficiency and its simplicity and flexibility of implementation. However, there are several areas that need close attention. For example, it is important to design the most specific and effective gRNAs to avoid off-target effects. The strategy that was employed here for maximizing specificity and avoiding off-target editing was verified by ultra deep sequencing of the whole genome and various other tests. The second issue relates to the controlled expression of Cas9 to avoid unnecessary presence of the protein that may non-specifically cause injury to the host genome in the long term and/or induce an immune response. The strategy here was developed for conditional expression of Cas, only in the presence of HIV-1 Tat, which provides a novel approach for activating and silencing gene editing for eradicating HIV-1 when the virus is on the rise.

The disclosures of each and every patent, patent application, and publication cited herein are hereby incorporated herein by reference in their entirety. One skilled in the art will readily appreciate that the present invention is well adapted to carry out the objects and obtain the ends and advantages mentioned, as well as those inherent therein. While the invention has been disclosed with reference to specific embodiments, it is apparent that other embodiments and variations of this invention may be devised by others skilled in the art without departing from the true spirit and scope used in the practice of the invention. The appended claims are intended to be construed to include all such embodiments and equivalent variations

What is claimed is:

1. A method of inactivating a human immunodeficiency virus (HIV) in vivo or in vitro, the method comprising administering to a subject or contacting a mammalian cell with a composition comprising an isolated nucleic acid sequence encoding a clustered regularly interspaced short palindromic repeats (CRISPR)-associated endonuclease operably linked to a truncated HIV long terminal repeat (LTR) promoter containing at least a core region and a trans activation response element (TAR) of a HIV LTR promoter.

2. The method of claim 1, wherein the isolated nucleic acid sequence further encodes at least one guide RNA that is complementary to a target nucleic acid sequence in HIV.

3. The method of claim 2, wherein the target nucleic acid sequence in HIV comprises a sequence within a coding region or non-coding region of HIV wherein the non-coding region comprises a long terminal repeat sequence of HIV.

4. (canceled)

5. The method of claim 3, wherein the target nucleic acid sequence comprises a sequence within the long terminal repeat of HIV, said sequence within the long terminal repeat of HIV comprises a sequence within the U3, R, or U5 regions that excludes any sequence of the truncated HIV LTR promoter.

6. (canceled)

7. The method of claim 1, wherein the mammalian cell is a latently infected cell.

8. The method of claim 7, wherein the latently infected cell is selected from the group consisting of: a CD4⁺ T cell, a macrophage, a monocyte, a gut-associated lymphoid cell, a microglial cell, and an astrocyte.

9. (canceled)

10. The method of claim 1, wherein the mammalian cell comprises a cultured cell from a subject having a HIV infection, a tissue explant, or a cell line.

11. The method of claim 1, wherein the CRISPR-associated endonuclease is Cas9.

12. The method of claim 1, wherein the CRISPR-associated endonuclease is optimized for expression in a human cell.

13. The method of claim 1, wherein the composition further comprises a sequence encoding a transactivating small RNA (tracrRNA).

14. The method of claim 13, wherein the tracrRNA is fused to a sequence encoding a guide RNA.

15. The method of claim 1, wherein the composition further comprises a sequence encoding a nuclear localization signal.

16. The method of claim 1, wherein the composition is operably linked to an expression vector, said expression vector is selected from the group consisting of: a lentiviral vector, an adenoviral vector, and an adeno-associated virus vector.

17. (canceled)

18. The method of claim 1, wherein the composition further comprises an enhancer region of the HIV-1 LTR promoter.

19. An isolated nucleic acid sequence comprising a sequence encoding a clustered regularly interspaced short palindromic repeats (CRISPR)-associated endonuclease operably linked to a truncated a human immunodeficiency virus (HIV) long terminal repeat (LTR) promoter containing at least a core region and a trans activation response element (TAR) of a HIV LTR promoter.

20. The isolated nucleic acid sequence of claim 19, wherein the sequence further encodes at least one guide RNA that is complementary to a target nucleic acid sequence in HIV.

21. The isolated nucleic acid sequence of claim 20, wherein the target nucleic acid sequence in HIV comprises a sequence within a coding region or a non-coding region of HIV, wherein the non-coding region comprises a long terminal repeat of HIV or a sequence within the long terminal repeat of HIV.

22. (canceled)

23. The isolated nucleic acid sequence of claim 21, wherein the sequence within the long terminal repeat of HIV comprises a sequence within the U3, R, or U5 regions that excludes any sequence of the truncated HIV LTR promoter.

24. The isolated nucleic acid sequence of claim 19, wherein the CRISPR-associated endonuclease is Cas9.

25. The isolated nucleic acid sequence of claim 19, wherein the CRISPR-associated endonuclease is optimized for expression in a human cell.

26. The isolated nucleic acid sequence of claim 19, further comprising a sequence encoding a transactivating small RNA (tracrRNA).

27. The isolated nucleic acid sequence of claim 26, wherein the tracrRNA is fused to a sequence encoding a guide RNA.

28. The isolated nucleic acid sequence of claim 19, further comprising a sequence encoding a nuclear localization signal.

29. The isolated nucleic acid sequence of claim 19, wherein the isolated nucleic acid sequence is operably linked to an expression vector, said expression vector is selected from the group consisting of: a lentiviral vector, an adenoviral vector, and an adeno-associated virus vector.

30. (canceled)

31. The isolated nucleic acid sequence of claim 19, further comprising an enhancer region of the HIV LTR promoter.

32. A pharmaceutical composition comprising a nucleic acid sequence encoding a clustered regularly interspaced short palindromic repeats (CRISPR)-associated endonuclease operably linked to a truncated human immunodeficiency virus (HIV) long terminal repeat (LTR) promoter containing at least a core region and a trans activation response element (TAR) of a HIV LTR promoter.

33. The pharmaceutical composition of claim 32, further comprising a pharmaceutically acceptable carrier.

34. The pharmaceutical composition of claim 33, wherein the pharmaceutically acceptable carrier comprises a lipid-based or polymer-based colloid.

35. The pharmaceutical composition of claim 34, wherein the colloid is a liposome, a hydrogel, a microparticle, a nanoparticle, or a block copolymer micelle.

36. The pharmaceutical composition of claim 32, wherein the pharmaceutical composition is formulated for topical application.

37. The pharmaceutical composition of claim 32, wherein the pharmaceutical composition is contained within a condom.

38. The pharmaceutical composition of claim 32, wherein the sequence further encodes at least one guide RNA that is complementary to a target nucleic acid sequence in HIV.

39. The pharmaceutical composition of claim 38, wherein the target nucleic acid sequence in HIV comprises a sequence within a coding region or a non-coding region of HIV, said non-coding region comprising a long terminal repeat of HIV.

40. (canceled)

41. The pharmaceutical composition of claim 39, wherein the target nucleic acid sequence comprises a sequence within the long terminal repeat of HIV, said sequence within the long terminal repeat of HIV comprises a sequence within the U3, R, or U5 regions that excludes any sequence of the truncated HIV-1 LTR promoter.

42. (canceled)

43. The pharmaceutical composition of claim 32, wherein the CRISPR-associated endonuclease is Cas9.

44. The pharmaceutical composition of claim 32, wherein the CRISPR-associated endonuclease is optimized for expression in a human cell.

45. The pharmaceutical composition of claim 32, further comprising a nucleic acid sequence encoding a transactivating small RNA (tracrRNA), wherein the tracrRNA is fused to a sequence encoding a guide RNA.

46. (canceled)

47. The pharmaceutical composition of claim 32, further comprising a sequence encoding a nuclear localization signal.

48. The pharmaceutical composition of claim 32, wherein the the isolated nucleic acid sequence is operably linked to an expression vector.

49. The pharmaceutical composition of claim 48, wherein the expression vector is selected from the group consisting of: a lentiviral vector, an adenoviral vector, and an adeno-associated virus vector.

50. The pharmaceutical composition of claim 32, wherein the sequence further encodes an enhancer region of the HIV-1 LTR promoter.

51. A method of treating a subject having a human immunodeficiency virus (HIV) infection, the method comprising administering to the subject a therapeutically effective amount of a pharmaceutical composition comprising a nucleic acid sequence encoding a clustered regularly interspaced short palindromic repeats (CRISPR)-associated endonuclease operably linked to a truncated HIV long terminal repeat (LTR) promoter containing at least a core region and a trans activation response element (TAR) of a HIV LTR promoter.

52. The method of treating the subject having the HIV infection of claim 51, wherein the sequence further encodes at least one guide RNA that is complementary to a target nucleic acid sequence in HIV.

53. The method of treating the subject having the HIV infection of claim 51, wherein HIV infection is a latent infection.

54. The method of treating the subject having the HIV infection of claim 51, wherein the pharmaceutical composition is administered topically or parenterally.

55. The method of treating the subject having the HIV infection of claim 51, further comprising administering an anti-retroviral agent.

56. The method of treating the subject having the HIV infection of claim 55, wherein the anti-retroviral agent is selected from the group consisting of non-nucleoside reverse transcriptase inhibitors, protease inhibitors, and entry inhibitors.

57. The method of treating the subject having the HIV infection of claim 56, wherein the anti-retroviral agent comprises highly active antiretroviral therapy.

58. The method of treating the subject having the HIV infection of claim 51, wherein the CRISPR-associated endonuclease is Cas9.

59. The method of treating the subject having the HIV infection of claim 57, wherein the pharmaceutical composition is operably linked to an expression vector.

60. The method of treating the subject having the HIV infection of claim 59, wherein the expression vector is selected from the group consisting of: a lentiviral vector, an adenoviral vector, and an adeno-associated virus vector.

61. The method of treating the subject having the HIV infection of claim 57, wherein the sequence further encodes an enhancer region of the HIV LTR promoter.

62.-93. (canceled)
